(19) United States
(12) Patent Application Publication
(10) Pub. No.: US 2008/0027082 A1
(43) Pub. Date: Jan. 31, 2008

(54) USE OF ADENOSINE A1 ANTAGONISTS IN RADIOCONTRAST MEDIA INDUCED NEPHROPATHY

(76) Inventors: Berthold Hocher, Hannover (DE); Yvan Fischer, Barsinghausen (DE); Klaus Witte, Hannover (DE); Dieter Ziegler, Hemmingen (DE)

Correspondence Address: MAYER BROWN LLP, P.O. BOX 2828, CHICAGO, IL, 60690, US

(21) Appl. No.: 11/765,290

(22) Filed: Jun. 19, 2007

Related U.S. Application Data
(60) Provisional application No. 60/805,173, filed on Jun. 19, 2006.
(60) Provisional application No. 60/805,168, filed on Jun. 19, 2006.
(60) Provisional application No. 60/871,062, filed on Dec. 20, 2006.

(51) Int. Cl. A61K 31/519; A61P 13/12

(52) U.S. Cl. 514/265.1

(57) ABSTRACT

Described herein are pharmaceutical combinations comprising a therapeutically effective amount of a first selective adenosine A1 antagonist and a first radiocontrast media. In one embodiment the selective adenosine A1 antagonist comprises 4-[(2-phenyl-7H-pyrrolo[2,3-d]pyrimidin-4-yl)amino]-trans-cyclohexanol methanesulfonate and/or (4S)-4-hydroxy-1-(2-phenyl-7H-pyrrolo[2,3-d]pyrimidin-4-yl)-L-prolinamide methanesulfonate. Also described are the use of a first selective adenosine A1 antagonist in the treatment of radiocontrast media induced nephropathy. Furthermore, a kit comprising a therapeutically effective amount of a first selective adenosine A1 antagonist and a first radiocontrast media is also described herein.

### CROSS REFERENCE TO RELATED APPLICATIONS

This application claims the benefit of U.S. Provisional Application Nos. 60/805,168 and 60/805,173 filed on Jun. 19, 2006 and U.S. Provisional Application No. 60/871,062 filed on Dec. 20, 2006 and all three are hereby incorporated by reference in their entirety to the extent permitted by law.

### FIELD

Pharmaceutical combinations comprising a therapeutically effective amount of a first selective adenosine A1 receptor antagonist and a first radiocontrast media (RM) are described herein. Also described is the use of said combinations for the treatment of radiocontrast media induced nephropathy as well as kits comprising said combinations.

### BACKGROUND

Interventional techniques, fast multislice computer tomographies and new 3D reconstruction techniques have increased the use of iodinated intravascular radiocontrast media (RM). The majority of examinations require iodinated RM for accurate and safe diagnosis and interventional procedures. Today approximately 60 million doses are given every year world wide (Andrew, 2004¹). The use of radiocontrast media can lead to a decline of excretory renal function that starts soon after administration. The renal dysfunction can be transient, persistent or even irreversible. Hence, the use of radiocontrast media has been associated with increased in-hospital morbidity, mortality, and cost of medical care and long admissions, especially in patients requiring dialysis. Radiocontrast media induced nephropathy (CIN) is therefore a clinically important problem.

CIN is the structural damage of the kidney. The definition of CIN varies. It can be defined as acute aggravation of renal functionality after application of RM, induced as proximate cause to the exclusion of alternative etiologies. The most common definition of a minor effect is an increase in serum creatinine greater than 25% or 44 mol/l (0.5 mg/dl) after the intravascular administration of a RM. A major effect is defined as increase in serum creatinine greater than 50% or 88 mmol/l (1 mg/dl). The pathogenesis of CIN is not fully understood. It is believed that two main factors, hemodynamic as well as tubular effects, are involved. Application of RM leads to a change in renal hemodynamics, manifesting itself as a decrease in the glomerular filtration rate (GFR). GFR is the rate of ultra filtration of plasma across the walls of the glomerular capillaries and measurement of total GFR of both kidneys provides a sensitive index of overall renal excretory function.

The glomerular filtration rate is calculated by comparing urine creatinine levels with the blood test results. A GFR value (see http://www.fpnotebook.com²) in a range of 97-137 ml/min/1.73 m² is adequate for a male human and of 88-128 ml/min/1.73 m is adequate for a female human, whereas a GFR lower than 15 ml/min/1.73 m² leads to kidney failure. A decrease in GFR induced by application of RM is considered to be the main cause for the development of CIN. Along the renal tubular system, substances like RM that are not reabsorbed become increasingly concentrated. Up to 99% of renal fluids are usually taken up by the action of manifold cellular and paracellular mechanisms. This means that the urine concentration of RM can increase by a factor of 100. Along with the continuous concentration process, tubular fluid containing RM will become increasingly viscous and can lead to tubular obstruction (Ueda, 1993³). Inevitably, intrarenal pressure increases as well, as the kidney cannot expand due to the surrounding capsule. As a consequence, renal perfusion pressure for the renal medulla may no longer be sufficient to allow for sufficient perfusion.

In the kidney, activation of A1AR in afferent glomerular arterioles has been suggested to contribute to tubuloglomerular feedback (TGF) which is a strategic feedback mechanism designed to control tubular flow and regional perfusion. The vasoconstriction elicited by elevations in [NaCl] in the macula densa region of the nephron. A role of adenosine in TGF response mediation is consistent with its effect to cause vasoconstriction. In addition to its vasoconstrictor effect, A₁ receptor stimulation contracts mesangial cells in the glomerulus (Olivera, 1989⁴). Acute renal failure caused by the injection of RM has been recognized for many years as a complication in diagnostic and interventional procedures. The incidence of acute renal failure directly induced by RM lies at approximately 10-15%, while the incidence of CIN defined by clinically significant increases in serum creatinine is as high as 22% (Porter, 1989⁵). The peak creatinine concentration occurs within 3-5 days of exposure to the contrast media and usually resolves satisfactorily. However, in about 10% of at-risk patients, dialysis is required. Preexisting renal insufficiency, reduced intravascular volume and additional underlying diseases (e.g. hypertension, diabetes mellitus) are thought to be some of the leading risk factors for radiocontrast media induced nephropathy. The osmolality, the measurement of the number of molecules and particles in a solution per kilogram of water, of the RM is regarded to be of great importance in radiocontrast induced nephropathy. The incidence of nephropathy induced by low-osmolar RM is low in the general population and has been calculated to be less than 2% (Nikolsky, 2003⁶).

Adenosine production is one of the discussed mechanisms behind CIN. Adenosine is an endogenous neuromodulator with predominantly inhibitory effects on the CNS, heart, kidneys and other organs. It is a naturally occurring nucleoside, which exerts its biological effects by interacting with a family of adenosine receptors known as A1, A2a, A2b, and A3, all of which modulate important physiological processes. Selective A1 adenosine receptor antagonists (A₁AR) have pronounced effects on the kidney and have shown to be potent diuretics and natriuretics with little effect on potassium excretion. Thus, they are renal protective and useful for the treatment of renal failure, renal dysfunction, nephritis, hypertension, and edema. The kidneys produce adenosine constitutively to regulate glomerular filtration and electrolyte reabsorption mediated by the adenosine A1 receptor system. The A1 adenosine receptor has been found to govern the vasoconstriction response of the afferent glomerular arteriole. Adenosine causes a reduction in the blood flow to the kidney, and thus a reduction in the glomerular filtration rate and the renal blood flow. Inhibition of the A1 receptor will heighten the glomerular filtration rate and correspondingly increase the rate of urine formation. The application of adenosine receptor antagonists has been implicated in protection from acute renal failure. The adenosine receptor antagonists aminophylline (combination of theophylline and ethylenediamine 2:1) and theophylline (which has been found to non-selectively antagonize adenosine receptors in the brain) were evaluated as potential agents to protect against radiocontrast media induced nephropathy (Shammas, 2001; Welch, 2002; Huber, 2002⁷). Aminophylline does not appear to add a protective role in preventing radiocontrast media induced nephropathy while theophylline was effective in preventing radiocontrast media induced nephropathy impaired renal excretory, endocrine and tubular function.

These results suggest that adenosine may play a role in the pathogenesis of CIN and that application of non-selective adenosine receptor antagonists has been implicated in protection from acute renal failure associated with RM treatment. Erley (1994⁸) investigated the influence of the non-selective adenosine antagonist theophylline on the glomerular filtration rate after the application of RM and determined that adenosine plays a major role in CIN. Furthermore, Arakawa (1996⁹) described the role of adenosine in the renal responses to the contrast medium iohexyl in dogs with and without pre-existing renal insufficiency. Arakawa indicated that in normal renal function, iohexyl elicits renal vasodilation by activating mainly the adenosine A2 receptors. Whereas in impaired renal function, iohexyl induces both A2 and A1 activation. Arakawa proposed that the adenosine A2 receptors were associated with the initial renal vasodilation and that the adenosine A1 receptors were responsible for the sustained aggravation of renal hemodynamics. Yao (2000¹⁰) investigated the influence of the selective adenosine A1 antagonist KW-3902 on radiocontrast media induced nephropathy in rats with chronic nitric oxide deficiency. Yao suggested adenosine influencing the pathogenesis of CIN via the activation of the A1 receptors. Greiner (2005¹¹) studied the influence on theophylline and acetylcystein separately and in combination on radiocontrast media induced nephropathy in intensive care patients and corroborated the prophylactic properties of theophylline in CIN. Lee (2006¹²) concluded that renal A1 adenosine receptors are only partially responsible in the pathogenesis of radiocontrast nephropathy. In experiments with renal A1 adenosine receptors knockout mice was found, that these mice are protected from acute renal failure induced by RM injection. Direct tubular toxicity seemed, however, not to be modulated by renal A1 adenosine receptors. Patent application EP 1 386 609 (CV Therapeutics¹³) described methods for restoring diuretic and renal function comprising adenosine A1 antagonist in combination with a diuretic. Patent application WO 99/31101 (Univ. South Florida¹⁴) discloses xanthine derivatives as adenosine A1 receptor antagonists. Additionally, radiolabelled derivatives and a method of imaging the adenosine A1 receptor antagonists for medical diagnostic purposes are mentioned.

### SUMMARY

Described herein is the use of a therapeutically effective amount of a first selective adenosine A1 receptor antagonist for the treatment of nephropathy induced by a first radiocontrast media.

Another embodiment described herein relates to a pharmaceutical combination comprising a therapeutically effective amount of a first selective adenosine A1 receptor antagonist and a radiocontrast media.

A further embodiment described herein relates to a kit comprising a therapeutically effective amount of a first selective adenosine A1 receptor antagonist and a radiocontrast media.

In an additional embodiment, the first A₁AR antagonist may be selected from the compounds represented by formula I 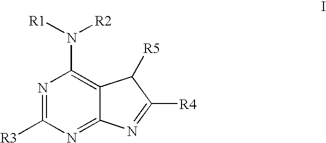
wherein
R1 and R2 are each independently selected from a hydrogen atom, an optionally substituted alkyl, optionally substituted aryl, or optionally substituted alkylaryl moiety or together form an optionally substituted heterocyclic ring;
R3 is selected from a hydrogen atom or an optionally substituted alkyl, optionally substituted aryl, or optionally substituted alkylaryl moiety;
R4 and R5 are each independently selected from a halogen atom, a hydrogen atom or an optionally substituted alkyl, optionally substituted aryl, or optionally substituted alkylaryl moiety, or R4 and R5 together form an optionally substituted heterocyclic or optionally substituted carbocyclic ring;
   and pharmaceutically acceptable salts of the foregoing, pharmaceutically acceptable prodrugs of the foregoing, and pharmaceutically acceptable solvates of the foregoing.

A further embodiment relates to a pharmaceutical combination comprising a combination of 4-[(2-phenyl-7H-pyrrolo[2,3-o]pyrimidin-4-yl)amino]-trans-cyclohexanol methanesulfonate or (4S)-4-hydroxy-1-(2-phenyl-7H-pyrrolo[2,3-o]pyrimidin-4-yl)-L-prolinamide methanesulfonate with a first RM.

In another embodiment the first RM may be an iodinated or gadolinium-based radiocontrast media selected from the group consisting of bunaiod, biligram, bilimiro, bilopaque, cholimil, ethiodol, diatrast, dionosil, falignost, gadobutrol, gadodiamide, gadopentetate dimeglumine, gastrografin, hexabrix, hippodin, mangafodipir, amidotrizoate, ethiodized oil, imagopaque, iodamide, iodipamide, iodixanol, iodophene, iophendylate, iomeron, iomeprol, iopamidol, iopanoic acid, iopiperidol, iophendylate, iopromide, iopydol, iosimenol, iothalamic acid, iotrolan, ioversol, ioxilan, ioxaglic acid, isopaque, ipodate, meglumine iothalamate, meglumine acetrizoate, meglumine diatrizoate, metrizamide, myelotrast, omnipaque, osbil, optiray, optojod, opacoron, perflutren, phenobutiodil, phentetiothalein sodium, priodax, propyliodone, skiodan, sodium iodomethamate, sodium diatrizoate, telepaque, teridax, tetrabrom, thorotrast, triognost, 1,3,5-Tri-n-hexyl-2,4,6-triiodobenzene, tyropanoate, visipaque or xenetix, pharmaceutically acceptable salts of the foregoing prodrugs of the foregoing, and a solvate of the foregoing.

### BRIEF DESCRIPTION OF THE FIGURES

FIG. 1: In volume restricted rats, hemodynamic measurements are made, and the TGF response is assessed.

FIG. 2: Experimental setting 2 is used for collecting urine. Diuresis, urine osmolality and urine viscosity are determined.

FIG. 3: Effects of Visipaque and substance 1 on renal cortical blood flow with measurements over a 20 min period following injection of Visipaque or vehicle (control) at time 0. Shown are means ±SEM (n=9), expressed as relative values compared to cortical flow rates recorded before Visipaque (or vehicle) challenge. *: P<0.05 Visipaque vs. Control; +: P<0.05 substance 1+Visipaque vs. Visipaque.

FIG. 4: Effects of Visipaque and substance 1 on renal cortical vascular conductance with measurements over 20 min period following injection of Visipaque or vehicle (control) at time 0. Shown are means ±SEM (n=9), expressed as relative values compared to cortical flow rates recorded before Visipaque (or vehicle) challenge. *: P<0.05 Visipaque vs. Control; +: P<0.05 substance 1+Visipaque vs. Visipaque.

FIG. 5: Effects of Visipaque and substance 1 on renal cortical oxygenation (PO₂) with measurements over 20 min period following injection of Visipaque or vehicle (control) at time 0. Shown are means ±SEM (n=9), expressed as relative values compared to cortical flow rates recorded before Visipaque (or vehicle) challenge. *: P<0.05 Visipaque vs. Control; +: P<0.05 substance 1+Visipaque vs. Visipaque.

### DESCRIPTION

Described herein is the use of a therapeutically effective amount of a first selective adenosine A1 antagonist for the prevention of nephropathy induced by a first radiocontrast media in mammals or humans. Also described herein is the use of a therapeutically effective amount of a first selective adenosine A1 antagonist of formula I 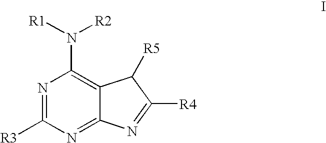
wherein
R1 and R2 are each independently selected from a hydrogen atom, an optionally substituted alkyl, optionally substituted aryl, or optionally substituted alkylaryl moiety or together form an optionally substituted heterocyclic ring;
R3 is selected from a hydrogen atom or an optionally substituted alkyl, optionally substituted aryl, or optionally substituted alkylaryl moiety;
R4 and R5 are each independently selected from a halogen atom, a hydrogen atom or an optionally substituted alkyl, optionally substituted aryl, or optionally substituted alkylaryl moiety, or R4 and R5 together form an optionally substituted heterocyclic or optionally substituted carbocyclic ring;
and pharmaceutically acceptable salts of the foregoing, pharmaceutically acceptable prodrugs of the foregoing, and pharmaceutically acceptable solvates of the foregoing,
for the prevention of nephropathy induced by a first radiocontrast media in mammals or humans.

One embodiment described herein relates to the use of a therapeutically effective amount of a first selective adenosine A1 antagonist for the prevention of an increase in serum creatinine levels induced by a first radiocontrast media in mammals or humans. A further embodiment described herein is the use of a therapeutically effective amount of a first selective adenosine A1 antagonist of formula I for the prevention of increase in serum creatinine levels induced by a first radiocontrast media in a transient, persistent or irreversible increase in serum creatinine levels induced by radiocontrast media in mammals or humans.

A further embodiment described herein relates to the use of a therapeutically effective amount of a first selective adenosine A1 antagonist for the prevention of decrease in renal blood flow induced by a first radiocontrast media. A further embodiment described herein is the use of a therapeutically effective amount of a first selective adenosine A1 antagonist of formula I for the prevention of decrease in renal blood flow induced by a first radiocontrast media in a transient, persistent or irreversible decrease in renal blood flow induced by radiocontrast media in mammals or humans.

A further embodiment described herein relates to the use of a therapeutically effective amount of a first selective adenosine A1 antagonist for preventing or reducing the risk or need of dialysis caused by radiocontrast media induced nephropathy which may be transient, persistent or irreversible, in mammals or humans. A further embodiment relates to the use of a therapeutically effective amount of a first selective adenosine A1 antagonist of formula I for preventing or reducing the risk or need of dialysis in a human or mammalian patient receiving radiocontrast media. In a further embodiment the need for dialysis is transient, persistent or irreversible.

A further embodiment relates to a pharmaceutical combination of a therapeutically effective amount of a first selective adenosine A1 antagonist and a first radiocontrast media, wherein the pharmaceutical combination is suitable for simultaneous, separate or step-wise administration to humans or mammals.

Another embodiment described herein relates to a kit comprising a therapeutically effective amount of a first selective adenosine A1 antagonist and a first radiocontrast media, wherein the pharmaceutical combination is suitable for simultaneous, separate or step-wise administration to humans or mammals.

An A1AR which can be used with the various embodiments described herein may be selected from formula I 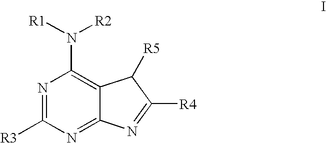
wherein
R1 and R2 are each independently selected from a hydrogen atom, an optionally substituted alkyl, optionally substituted aryl, or optionally substituted alkylaryl moiety or together form an optionally substituted heterocyclic ring; R3 is selected from a hydrogen atom or an optionally substituted alkyl, optionally substituted aryl, or optionally substituted alkylaryl moiety; R4 and R5 are each independently selected from a halogen atom, a hydrogen atom or an optionally substituted alkyl, optionally substituted aryl, or optionally substituted alkylaryl moiety, or R4 and R5 together form an optionally substituted heterocyclic or optionally substituted carbocyclic ring; in one embodiment
R1 and R2 are each independently selected from a hydrogen atom, an optionally substituted alkyl or together form an optionally substituted heterocyclic ring; R3 is a hydrogen atom or an optionally substituted aryl; R4 and R5 are each independently selected from a halogen atom or a hydrogen atom;
in a further embodiment
R1 is a hydrogen and R2 is an optionally substituted cyclohexyl ring, or R1 and R2 together form an optionally substituted pyrrolidine ring; R3 is a phenyl ring; R4 and R5 are each a hydrogen atom;
In a further embodiment, the compounds of formula can be in the form of pharmaceutically acceptable salts of the foregoing, pharmaceutically acceptable prodrugs of the foregoing, and pharmaceutically acceptable solvates of the foregoing.

In a further embodiment, A1ARs described herein may be selected from 4-[(2-phenyl-7H-pyrrolo[2,3-a]pyrimidin-4-yl)amino]-trans-cyclohexanol methanesulfonate or (4S)-4-hydroxy-1-(2-phenyl-7H-pyrrolo[2,3-d]pyrimidin-4-yl)-L-prolinamide methanesulfonate as well as pharmaceutically acceptable salts of the foregoing, pharmaceutically acceptable prodrugs of the foregoing, and pharmaceutically acceptable solvates of the foregoing.

Additional A1ARs suitable for use herein are described within the international patent applications WO 99/62518, WO 01/39777, WO 02/057267 and WO 2004/094428 (Osi Pharmaceuticals and Solvay Pharmaceuticals¹⁵).

Suitable RM which can be used as described herein include iodinated or gadolinium-based radiocontrast media selected from the group consisting of bunaiod, biligram, bilimiro, bilopaque, cholimil, ethiodol, diatrast, dionosil, falignost, gadobutrol, gadodiamide, gadopentetate dimeglumine, gastrografin, hexabrix, hippodin, mangafodipir, amidotrizoate, ethiodized oil, imagopaque, iodamide, iodipamide, iodixanol, iodophene, iophendylate, iomeron, iomeprol, iopamidol, iopanoic acid, iopiperidol, iophendylate, iopromide, iopydol, iosimenol, iothalamic acid, iotrolan, ioversol, ioxilan, ioxaglic acid, isopaque, ipodate, meglumine iothalamate, meglumine acetrizoate, meglumine diatrizoate, metrizamide, myelotrast, omnipaque, osbil, optiray, optojod, opacoron, perflutren, phenobutiodil, phentetiothalein sodium, priodax, propyliodone, skiodan, sodium iodomethamate, sodium diatrizoate, telepaque, teridax, tetrabrom, thorotrast, triognost, 1,3,5-Tri-n-hexyl-2,4,6-triiodobenzene, tyropanoate, visipaque, xenetix, pharmaceutically acceptable salts, prodrugs and solvates of the foregoing.

In an embodiment the RM includes xenetix, omnipaque or visipaque.

Some examples (Schering, Bracco Industria Chimica, Univ. California, Nyegaard, Cook Imaging Corporation, Mallinckrodt, Eprova, Nycomed and Savag¹⁶) of additional RM suitable for use herein are described in EP 0 022 744, EP 0 023 992, EP 0 026 281, EP 0 033 426, EP 0 108 638, EP 0 317 492, WO 87/00757, WO 89/08101, U.S. Pat. No. 2,776,241, U.S. Pat. No. 3,290,366, U.S. Pat. No. 3,360,436, U.S. Pat. No. 5,349,085, GB 1 321 591, DE 2 547 789, DE 2 726 196 and DE 2 909 439. The foregoing examples of suitable RM are meant to be illustrative and not to limit the group of suitable RM.

Fore ease of reference, 4-[(2-phenyl-7H-pyrrolo[2,3-o]pyrimidin-4-yl)amino]-trans-cyclohexanol methanesulfonate will hereafter be referred to as substance 1 and (4S)-4-hydroxy-1-(2-phenyl-7H-pyrrolo[2,3-a]pyrimidin-4-yl)-L-prolinamide methanesulfonate will be referred to as substance 2.

A further embodiment described herein relates to a use of substance 1 and bunaiod, or substance 1 and biligram, or substance 1 and bilimiro, or substance 1 and bilopaque, or substance 1 and cholimil, or substance 1 and ethiodol, or substance 1 and diatrast, or substance 1 and dionosil, or substance 1 and falignost, or substance 1 and gadobutrol, or substance 1 and gadodiamide, or substance 1 and gadopentetate dimeglumine, or substance 1 and gastrografin, or substance 1 and hexabrix, or substance 1 and hippodin, or substance 1 and mangafodipir, or substance 1 and amidotrizoate, or substance 1 and ethiodized oil, or substance 1 and imagopaque, or substance 1 and iodamide, or substance 1 and iodipamide, or substance 1 and iodixanol, or substance 1 and iodophene, or substance 1 and iophendylate, or substance 1 and iomeron, or substance 1 and iomeprol, or substance 1 and iopamidol, or substance 1 and iopanoic acid, or substance 1 and iopiperidol, or substance 1 and iophendylate, or substance 1 and iopromide, or substance 1 and iopydol, or substance 1 and iosimenol, or substance 1 and iothalamic acid, or substance 1 and iotrolan, or substance 1 and ioversol, or substance 1 and ioxilan, or substance 1 and ioxaglic acid, or substance 1 and isopaque, or substance 1 and ipodate, or substance 1 and meglumine iothalamate, or substance 1 and meglumine acetrizoate, or substance 1 and meglumine diatrizoate, or substance 1 and metrizamide, or substance 1 and myelotrast, or substance 1 and omnipaque, or substance 1 and osbil, or substance 1 and optiray, or substance 1 and optojod, or substance 1 and opacoron, or substance 1 and perflutren, or substance 1 and phenobutiodil, or substance 1 and phentetiothalein sodium, or substance 1 and priodax, or substance 1 and propyliodone, or substance 1 and skiodan, or substance 1 and sodium iodomethamate, or substance 1 and sodium diatrizoate, or substance 1 and telepaque, or substance 1 and teridax, or substance 1 and tetrabrom, or substance 1 and thorotrast, or substance 1 and triognost, or substance 1 and 1,3,5-Tri-n-hexyl-2,4,6-triiodobenzene, or substance 1 and tyropanoate, or substance 1 and visipaque, or substance 1 and xenetix. A further embodiment described herein relates to a pharmaceutical combination of substance 1 and bunaiod, or substance 1 and biligram, or substance 1 and bilimiro, or substance 1 and bilopaque, or substance 1 and cholimil, or substance 1 and ethiodol, or substance 1 and diatrast, or substance 1 and dionosil, or substance 1 and falignost, or substance 1 and gadobutrol, or substance 1 and gadodiamide, or substance 1 and gadopentetate dimeglumine, or substance 1 and gastrografin, or substance 1 and hexabrix, or substance 1 and hippodin, or substance 1 and mangafodipir, or substance 1 and amidotrizoate, or substance 1 and ethiodized oil, or substance 1 and imagopaque, or substance 1 and iodamide, or substance 1 and iodipamide, or substance 1 and iodixanol, or substance 1 and iodophene, or substance 1 and iophendylate, or substance 1 and iomeron, or substance 1 and iomeprol, or substance 1 and iopamidol, or substance 1 and iopanoic acid, or substance 1 and iopiperidol, or substance 1 and iophendylate, or substance 1 and iopromide, or substance 1 and iopydol, or substance 1 and iosimenol, or substance 1 and iothalamic acid, or substance 1 and iotrolan, or substance 1 and ioversol, or substance 1 and ioxilan, or substance 1 and ioxaglic acid, or substance 1 and isopaque, or substance 1 and ipodate, or substance 1 and meglumine iothalamate, or substance 1 and meglumine acetrizoate, or substance 1 and meglumine diatrizoate, or substance 1 and metrizamide, or substance 1 and myelotrast, or substance 1 and omnipaque, or substance 1 and osbil, or substance 1 and optiray, or substance 1 and optojod, or substance 1 and opacoron, or substance 1 and perflutren, or substance 1 and phenobutiodil, or substance 1 and phentetiothalein sodium, or substance 1 and priodax, or substance 1 and propyliodone, or substance 1 and skiodan, or substance 1 and sodium iodomethamate, or substance 1 and sodium diatrizoate, or substance 1 and telepaque, or substance 1 and teridax, or substance 1 and tetrabrom, or substance 1 and thorotrast, or substance 1 and triognost, or substance 1 and 1,3,5-Tri-n-hexyl-2,4,6-triiodobenzene, or substance 1 and tyropanoate, or substance 1 and visipaque, or substance 1 and xenetix. A further embodiment described herein relates to a kit comprising substance 1 and bunaiod, or substance 1 and biligram, or substance 1 and bilimiro, or substance 1 and bilopaque, or substance 1 and cholimil, or substance 1 and ethiodol, or substance 1 and diatrast, or substance 1 and dionosil, or substance 1 and falignost, or substance 1 and gadobutrol, or substance 1 and gadodiamide, or substance 1 and gadopentetate dimeglumine, or substance 1 and gastrografin, or substance 1 and hexabrix, or substance 1 and hippodin, or substance 1 and mangafodipir, or substance 1 and amidotrizoate, or substance 1 and ethiodized oil, or substance 1 and imagopaque, or substance 1 and iodamide, or substance 1 and iodipamide, or substance 1 and iodixanol, or substance 1 and iodophene, or substance 1 and iophendylate, or substance 1 and iomeron, or substance 1 and iomeprol, or substance 1 and iopamidol, or substance 1 and iopanoic acid, or substance 1 and iopiperidol, or substance 1 and iophendylate, or substance 1 and iopromide, or substance 1 and iopydol, or substance 1 and iosimenol, or substance 1 and iothalamic acid, or substance 1 and iotrolan, or substance 1 and ioversol, or substance 1 and ioxilan, or substance 1 and ioxaglic acid, or substance 1 and isopaque, or substance 1 and ipodate, or substance 1 and meglumine iothalamate, or substance 1 and meglumine acetrizoate, or substance 1 and meglumine diatrizoate, or substance 1 and metrizamide, or substance 1 and myelotrast, or substance 1 and omnipaque, or substance 1 and osbil, or substance 1 and optiray, or substance 1 and optojod, or substance 1 and opacoron, or substance 1 and perflutren, or substance 1 and phenobutiodil, or substance 1 and phentetiothalein sodium, or substance 1 and priodax, or substance 1 and propyliodone, or substance 1 and skiodan, or substance 1 and sodium iodomethamate, or substance 1 and sodium diatrizoate, or substance 1 and telepaque, or substance 1 and teridax, or substance 1 and tetrabrom, or substance 1 and thorotrast, or substance 1 and triognost, or substance 1 and 1,3,5-Tri-n-hexyl-2,4,6-triiodobenzene, or substance 1 and tyropanoate, or substance 1 and visipaque, or substance 1 and xenetix.

A further embodiment described herein relates to a use of substance 2 and bunaiod, or substance 2 and biligram, or substance 2 and bilimiro, or substance 2 and bilopaque, or substance 2 and cholimil, or substance 2 and ethiodol, or substance 2 and diatrast, or substance 2 and dionosil, or substance 2 and falignost, or substance 2 and gadobutrol, or substance 2 and gadodiamide, or substance 2 and gadopentetate dimeglumine, or substance 2 and gastrografin, or substance 2 and hexabrix, or substance 2 and hippodin, or substance 2 and mangafodipir, or substance 2 and amidotrizoate, or substance 2 and ethiodized oil, or substance 2 and imagopaque, or substance 2 and iodamide, or substance 2 and iodipamide, or substance 2 and iodixanol, or substance 2 and iodophene, or substance 2 and iophendylate, or substance 2 and iomeron, or substance 2 and iomeprol, or substance 2 and iopamidol, or substance 2 and iopanoic acid, or substance 2 and iopiperidol, or substance 2 and iophendylate, or substance 2 and iopromide, or substance 2 and iopydol, or substance 2 and iosimenol, or substance 2 and iothalamic acid, or substance 2 and iotrolan, or substance 2 and ioversol, or substance 2 and ioxilan, or substance 2 and loxaglic acid, or substance 2 and isopaque, or substance 2 and ipodate, or substance 2 and megiumine iothalamate, or substance 2 and meglumine acetrizoate, or substance 2 and meglumine diatrizoate, or substance 2 and metrizamide, or substance 2 and myelotrast, or substance 2 and omnipaque, or substance 2 and osbil, or substance 2 and optiray, or substance 2 and optojod, or substance 2 and opacoron, or substance 2 and perflutren, or substance 2 and phenobutiodil, or substance 2 and phentetiothalein sodium, or substance 2 and priodax, or substance 2 and propyliodone, or substance 2 and skiodan, or substance 2 and sodium iodomethamate, or substance 2 and sodium diatrizoate, or substance 2 and telepaque, or substance 2 and teridax, or substance 2 and tetrabrom, or substance 2 and thorotrast, or substance 2 and triognost, or substance 2 and 1,3,5-Tri-n-hexyl-2,4,6-triiodobenzene, or substance 2 and tyropanoate, or substance 2 and visipaque, or substance 2 and xenetix. A further embodiment described herein relates to a pharmaceutical combination of substance 2 and bunaiod, or substance 2 and biligram, or substance 2 and bilimiro, or substance 2 and bilopaque, or substance 2 and cholimil, or substance 2 and ethiodol, or substance 2 and diatrast, or substance 2 and dionosil, or substance 2 and falignost, or substance 2 and gadobutrol, or substance 2 and gadodiamide, or substance 2 and gadopentetate dimeglumine, or substance 2 and gastrografin, or substance 2 and hexabrix, or substance 2 and hippodin, or substance 2 and mangafodipir, or substance 2 and amidotrizoate, or substance 2 and ethiodized oil, or substance 2 and imagopaque, or substance 2 and iodamide, or substance 2 and iodipamide, or substance 2 and iodixanol, or substance 2 and iodophene, or substance 2 and iophendylate, or substance 2 and iomeron, or substance 2 and iomeprol, or substance 2 and iopamidol, or substance 2 and iopanoic acid, or substance 2 and iopiperidol, or substance 2 and iophendylate, or substance 2 and iopromide, or substance 2 and iopydol, or substance 2 and iosimenol, or substance 2 and iothalamic acid, or substance 2 and iotrolan, or substance 2 and ioversol, or substance 2 and ioxilan, or substance 2 and ioxaglic acid, or substance 2 and isopaque, or substance 2 and ipodate, or substance 2 and meglumine iothalamate, or substance 2 and meglumine acetrizoate, or substance 2 and meglumine diatrizoate, or substance 2 and metrizamide, or substance 2 and myelotrast, or substance 2 and omnipaque, or substance 2 and osbil, or substance 2 and optiray, or substance 2 and optojod, or substance 2 and opacoron, or substance 2 and perflutren, or substance 2 and phenobutiodil, or substance 2 and phentetiothalein sodium, or substance 2 and priodax, or substance 2 and propyliodone, or substance 2 and skiodan, or substance 2 and sodium iodomethamate, or substance 2 and sodium diatrizoate, or substance 2 and telepaque, or substance 2 and teridax, or substance 2 and tetrabrom, or substance 2 and thorotrast, or substance 2 and triognost, or substance 2 and 1,3,5-Tri-n-hexyl-2,4,6-triiodobenzene, or substance 2 and tyropanoate, or substance 2 and visipaque, or substance 2 and xenetix. A further embodiment described herein relates to a kit comprising substance 2 and bunaiod, or substance 2 and biligram, or substance 2 and bilimiro, or substance 2 and bilopaque, or substance 2 and cholimil, or substance 2 and ethiodol, or substance 2 and diatrast, or substance 2 and dionosil, or substance 2 and falignost, or substance 2 and gadobutrol, or substance 2 and gadodiamide, or substance 2 and gadopentetate dimeglumine, or substance 2 and gastrografin, or substance 2 and hexabrix, or substance 2 and hippodin, or substance 2 and mangafodipir, or substance 2 and amidotrizoate, or substance 2 and ethiodized oil, or substance 2 and imagopaque, or substance 2 and iodamide, or substance 2 and iodipamide, or substance 2 and iodixanol, or substance 2 and iodophene, or substance 2 and iophendylate, or substance 2 and iomeron, or substance 2 and iomeprol, or substance 2 and iopamidol, or substance 2 and iopanoic acid, or substance 2 and iopiperidol, or substance 2 and iophendylate, or substance 2 and iopromide, or substance 2 and iopydol, or substance 2 and iosimenol, or substance 2 and iothalamic acid, or substance 2 and iotrolan, or substance 2 and ioversol, or substance 2 and ioxilan, or substance 2 and ioxaglic acid, or substance 2 and isopaque, or substance 2 and ipodate, or substance 2 and meglumine iothalamate, or substance 2 and meglumine acetrizoate, or substance 2 and meglumine diatrizoate, or substance 2 and metrizamide, or substance 2 and myelotrast, or substance 2 and omnipaque, or substance 2 and osbil, or substance 2 and optiray, or substance 2 and optojod, or substance 2 and opacoron, or substance 2 and perflutren, or substance 2 and phenobutiodil, or substance 2 and phentetiothalein sodium, or substance 2 and priodax, or substance 2 and propyliodone, or substance 2 and skiodan, or substance 2 and sodium iodomethamate, or substance 2 and sodium diatrizoate, or substance 2 and telepaque, or substance 2 and teridax, or substance 2 and tetrabrom, or substance 2 and thorotrast, or substance 2 and triognost, or substance 2 and 1,3,5-Tri-n-hexyl-2,4,6-triiodobenzene, or substance 2 and tyropanoate, or substance 2 and visipaque, or substance 2 and xenetix.

The term “therapeutically effective amount” of a drug or pharmacologically active agent means a nontoxic but sufficient amount of the drug or active agent needed to provide the desired effect. In the combination therapy described herein, a “therapeutically effective amount” of one component of the combination is the amount of that compound that is effective to provide the desired effect when used in combination with the other components of the combination. The amount that is “effective” will vary from subject to subject, depending on the species, age, general condition of the individual, the particular active agent or agents, and the like. It thus is not always possible to specify an exact “therapeutically effective amount”. However, an appropriate “therapeutically effective amount” in any individual case may be determined by a person of ordinary skill in the art.

In general, the first RM is not administered until the plasma level of the first selective adenosine A1 receptor antagonist has reached a concentration of about 10 ng/ml to about 500 ng/ml. Also described herein are all concentration or concentration ranges, which lie within the range of 10 ng/ml to 500 ng/ml. In one embodiment, the first selective adenosine A1 antagonist has a concentration of about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, and about 500 ng/ml, and any other concentration or concentration ranges, which lie within in any ranges defined by two of the previously mentioned concentration values, where the lower limit of said range is defined by the lower value and the upper limit of said range by the higher value, e.g. a range of about 110 to about 180 ng/ml, about 370 to about 390 ng/ml, about 10 to about 150 ng/ml, etc. A further embodiment described herein includes the use of a first radiocontrast media which is not administered until the therapeutically effective amount of the first selective adenosine A1 receptor antagonist is sufficient to provide a plasma level concentration of about 10 ng/ml to about 500 ng/ml, about 20 ng/ml to about 400 ng/ml or about 30 ng/ml to about 300 ng/ml. A further embodiment includes a pharmaceutical combination comprising the first radiocontrast media which is not administered until the therapeutically effective amount of the first selective adenosine A1 receptor antagonist is sufficient to provide a plasma level concentration of about 10 ng/ml to about 500 ng/ml, about 20 ng/ml to about 400 ng/ml or about 30 ng/ml to about 300 ng/ml. A further embodiment includes using the first radiocontrast media which is not administered until the therapeutically effective amount of the first selective adenosine A1 receptor antagonist is sufficient to provide a plasma level concentration of about 10 ng/ml to about 500 ng/ml, about 20 ng/ml to about 400 ng/ml or about 30 ng/ml to about 300 ng/ml. A further embodiment also includes a pharmaceutical combination comprising the first radiocontrast media which is not administered until the therapeutically effective amount of said a first selective adenosine A1 receptor antagonist is sufficient to provide a plasma level concentration of about 10 ng/ml to about 500 ng/ml, about 20 ng/ml to about 400 ng/ml or about 30 ng/ml to about 300 ng/ml.

The duration of administration of the maintenance dosage of the first selective A1 adenosine antagonist is that which is sufficient to maintain the plasma level of the first selective A1 adenosine antagonist at a concentration of between about 10 ng/ml and about 500 ng/ml. The amount of the first selective A1 adenosine antagonist to be administered to reach and maintain a specific plasma level of the first selective A1 adenosine antagonist corresponds to specific dosages to be administered to a patient. The skilled artisan is able to select an appropriate dosage for a specific patient. A further embodiment also includes every concentration or concentration range which lies within the range of between 10 ng/ml to 500 ng/ml. In a further embodiment, the first selective adenosine A1 antagonist has a concentration of about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, and about 500 ng/ml, and every concentration or concentration range which lies in any ranges defined by two of the before mentioned concentration values, where the lower limit of said range is defined by the minor value and the upper limit of said range by the higher value, e.g. a range of about 10 ng/ml to about 80 ng/ml, about 320 ng/ml to about 390 ng/ml, about 100 ng/ml to about 50 ng/ml, etc.

The duration of administration of the maintenance dosage of the first selective A1 adenosine antagonist lies between about 0.1 hours and about 48 hours to maintain the plasma level of the first selective A1 adenosine at a concentration of between about 10 ng/ml to about 500 ng/ml. A further embodiment also includes every time interval which lies within the time period of about 0.1 hours and about 48 hours. In a further embodiment, the time period of administration of the maintenance dosage is about 0.1, about 0.3, about 0.5, about 1, about 1.5, about 2, about 2.5, about 3, about 3.5, about 4, about 4.5, about 5, about 5.5 about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, about 27, about 27.5, about 28, about 28.5, about 29, about 29.5, about 30, about 30.5, about 31, about 31.5, about 32, about 32.5, about 33, about 33.5, about 34, about 34.5, about 35, about 35.5, about 36, about 36.5, about 37, about 37.5, about 38, about 38.5, about 39, about 39.5, about 40, about 40.5, about 41, about 41.5, about 42, about 42.5, about 43, about 43.5, about 44, about 44.5, about 45, about 45.5, about 46, about 46.5, about 47, about 47.5 and about 48 hours, and every time period which lies in any ranges defined by two of the before mentioned values, where the lower limit of said range is defined by the minor value and the upper limit of said range by the higher value, e.g. a range of about 1 to about 2 hours, about 0.1 to about 10 hours, about 0.2 to about 6 hours, about 2 hours to about 45 hours, about 9.5 to about 35 hours, etc.

The first selective adenosine A1 receptor antagonist may be administered intravenously in a loading dose followed by one or more maintenance doses. The first selective adenosine A1 receptor antagonist loading dose is administered at a time period of between about 5 and about 25 minutes prior to the administration of the first radiocontrast media and the maintenance dosage of the first selective adenosine A1 receptor antagonist is administered over a period of up to 48 hours subsequent to administration of the loading dose of the first selective A1 receptor antagonist. In a further embodiment the maintenance dose is administered over a period of up to about 0.1, about 0.3, about 0.5, about 1, about 1.5, about 2, about 2.5, about 3, about 3.5, about 4, about 4.5, about 5, about 5.5 about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, about 27, about 27.5, about 28, about 28.5, about 29, about 29.5, about 30, about 30.5, about 31, about 31.5, about 32, about 32.5, about 33, about 33.5, about 34, about 34.5, about 35, about 35.5, about 36, about 36.5, about 37, about 37.5, about 38, about 38.5, about 39, about 39.5, about 40, about 40.5, about 41, about 41.5, about 42, about 42.5, about 43, about 43.5, about 44, about 44.5, about 45, about 45.5, about 46, about 46.5, about 47, about 47.5 and about 48 hours. A further embodiment includes every time interval which lies within time period of about 5 minutes to 25 minutes prior to the administration of the first radiocontrast media and the maintenance dose of the first selective adenosine A1 receptor antagonist is administered over a period of up to about 48 hours subsequent to administration of the loading dose of the first selective A1 receptor antagonist. In a further embodiment the maintenance dose is administered over a period of up to about 0.1, about 0.3, about 0.5, about 1, about 1.5, about 2, about 2.5, about 3, about 3.5, about 4, about 4.5, about 5, about 5.5 about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, about 27, about 27.5, about 28, about 28.5, about 29, about 29.5, about 30, about 30.5, about 31, about 31.5, about 32, about 32.5, about 33, about 33.5, about 34, about 34.5, about 35, about 35.5, about 36, about 36.5, about 37, about 37.5, about 38, about 38.5, about 39, about 39.5, about 40, about 40.5, about 41, about 41.5, about 42, about 42.5, about 43, about 43.5, about 44, about 44.5, about 45, about 45.5, about 46, about 46.5, about 47, about 47.5 and about 48 hours. In a further embodiment the first selective adenosine A1 receptor antagonist may be administered intravenously at about 5, about 6, about 7, about 8, about 9, about 10, about 11, about 12, about 13, about 14, about 15, about 16, about 17, about 18, about 19, about 20, about 21, about 22, about 23, about 24 and about 25 minutes, and every period which lies in any ranges defined by two of the before mentioned values, where the lower limit of said range is defined by the minor value and the upper limit of said range by the upper value, e.g. a range of about 10 minutes to about 18 minutes, about 20 minutes to about 25 minutes, about 12 minutes to about 15 minutes, etc., prior to the administration of the first radiocontrast media, and the maintenance dosage of the first selective adenosine A1 receptor antagonist is administered over a period of up to about 48 hours subsequent to administration of the loading dose of the first selective A1 receptor antagonist. In a further embodiment the maintenance dose is administered over a period of up to about 0.1, about 0.3, about 0.5, about 1, about 1.5, about 2, about 2.5, about 3, about 3.5, about 4, about 4.5, about 5, about 5.5, about 6, about 6.5, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, about 27, about 27.5, about 28, about 28.5, about 29, about 29.5, about 30, about 30.5, about 31, about 31.5, about 32, about 32.5, about 33, about 33.5, about 34, about 34.5, about 35, about 35.5, about 36, about 36.5, about 37, about 37.5, about 38, about 38.5, about 39, about 39.5, about 40, about 40.5, about 41, about 41.5, about 42, about 42.5, about 43, about 43.5, about 44, about 44.5, about 45, about 45.5, about 46, about 46.5, about 47, about 47.5 and about 48 hours.

A further embodiment includes a use comprising the therapeutically effective amount of the first selective adenosine A1 receptor antagonist in a loading dose to be administered intravenously followed by a maintenance dose. The loading does of the first selective adenosine A1 receptor antagonist is to be administered at a time period of between about 5 and about 25 minutes, between about 10 and about 20 minutes, between about 13 and about 17 minutes, or about 15 minutes prior to the administration of the first radiocontrast media. The maintenance dose of the first selective adenosine A1 receptor antagonist is administered over a period of up to about 48 hours subsequent to administration of the loading dose of the first selective A1 receptor antagonist. A further embodiment includes a pharmaceutical combination comprising the therapeutically effective amount of the first selective adenosine A1 receptor antagonist in a loading dose which is administered intravenously followed by a maintenance dose, where the first selective adenosine A1 receptor antagonist loading dose is administered at a time period of about 5 to about 25 minutes, about 10 to about 20 minutes, about 13 to about 17 minutes, or about 15 minutes prior to the administration of the first radiocontrast media. The maintenance dose of the first selective adenosine A1 receptor antagonist is administered over a period of up to 48 hours subsequent to administration of the loading dosage of the first selective A1 receptor antagonist.

A further embodiment described herein is a kit comprising the therapeutically effective amount of the first selective adenosine A1 receptor antagonist in a loading dose to be administered intravenously followed by a maintenance dose where the first selective adenosine A1 receptor antagonist loading dose is administered at a time period of between about 5 and about 25 minutes, between about 10 and about 20 minutes, between about 13 and about 17 minutes, or about 15 minutes prior to the administration of the first radiocontrast media. The maintenance dose of the first selective adenosine A1 receptor antagonist is administered over a period of up to about 48 hours subsequent to administration of the loading dose of the first selective A1 receptor antagonist. The kit may also include a first radiocontrast media.

The embodiments described herein are not limited to specific dosage forms, carriers, excipients, or the like, as such may vary. It is also to be understood that the terminology used herein is for the purpose of describing particular embodiments only, and is not intended to be limiting.

It must be noted that as used in this specification and the appended claims, the singular forms “a”, “an”, and “the” include plural referents unless the context clearly dictates otherwise. Thus, for example, reference to “a therapeutically effective agent” includes a single agent as well as two or more different agents in combination, and reference to “a carrier” includes mixtures of two or more carriers as well as a single carrier, and the like.

The terms “A1AR”, “selective adenosine A1 antagonist” and “selective adenosine A1 receptor antagonist” are used interchangeably herein to refer to a chemical compound that induces a desired pharmacological and physiological effect.

The first selective adenosine A1 antagonist may be administered orally and/or intravenously. One embodiment included here is the use of a therapeutically effective amount of the first selective adenosine A1 receptor antagonist to be administered orally, such as an extended release formulation, prior to the administration of the first radiocontrast agent. A further embodiment included herein is the use of a therapeutically effective amount of the first selective adenosine A1 receptor antagonist in a loading dose which is administered intravenously followed by a maintenance dose where the first selective adenosine A1 receptor antagonist loading dose is administered at a time period of between about 5 and about 25 minutes, between about 10 and about 20 minutes, between about 13 and about 17 minutes, or about 15 minutes prior to the administration of the first radiocontrast media. The maintenance dose of the first selective adenosine A1 receptor antagonist is administered over a period of up to about 48 hours subsequent to administration of the loading dose of the first selective A1 receptor antagonist.

A further embodiment includes a pharmaceutical combination comprising a therapeutically effective amount of the first selective adenosine A1 receptor antagonist to be administered orally, such as in an extended release formulation, prior to the administration of the first radiocontrast agent. The pharmaceutical combination may also include a first radiocontrast media.

A further embodiment includes a pharmaceutical combination comprising a therapeutically effective amount of the first selective adenosine A1 receptor antagonist in a loading dose which is administered intravenously followed by a maintenance dose where the first selective adenosine A1 receptor antagonist loading dose is administered at a time period of about 5 to about 25 minutes, between about 10 and about 20 minutes, between about 13 and about 17 minutes, or about 15 minutes prior to the administration of the first radiocontrast media. The maintenance dose of the first selective adenosine A1 receptor antagonist is administered over a period of up to about 48 hours subsequent to administration of the loading dose of the first selective A1 receptor antagonist. The pharmaceutical combination may also include a first radiocontrast media.

A further embodiment disclosed herein also includes a kit comprising the therapeutically effective amount of the first selective adenosine A1 receptor antagonist to be administered orally, such as in an extended release formulation, prior to the administration of the first radiocontrast agent. The pharmaceutical combination may also include a first radiocontrast media.

A further embodiment includes a kit comprising the therapeutically effective amount of the first selective adenosine A1 receptor antagonist in a loading dose to be administered intravenously followed by a maintenance dose where the first selective adenosine A1 receptor antagonist loading dose is administered at a time period of between 5 and about 25 minutes, between about 10 and about 20 minutes, between about 13 and about 17 minutes, or about 15 minutes prior to the administration of the first radiocontrast media. The maintenance dose of the first selective adenosine A1 receptor antagonist is administered over a period of up to about 48 hours subsequent to administration of the loading dose of the first selective A1 receptor antagonist. The pharmaceutical combination may also include a first radiocontrast media.

The term “intravenously” relates to parenteral application and includes injection or infusion into a vein or an artery, without limiting the group of parenteral application forms.

The term “orally” relates to enteral application which includes application of e.g. tablets, drops, pills, capsules, pellets, granules, etc. by mouth, without limiting the group of enteral application forms.

“Extended release” refers to a pharmaceutical dosage form. The term “extended” includes e.g. “prolonged”, “retarded”, “controlled”, “retentive” and “delayed” dosage forms, without limiting.

The term “container” refers to a hermetically sealed storage box for pharmaceuticals. It includes storage boxes for fluid pharmaceuticals as e.g. ampoules, vials, flask, dispensers, syringes, etc. as well as storage boxes for solid pharmaceuticals as e.g. blisters, capsules, etc. without limiting the group of storage boxes.

The term “irreversible” as used herein can be used interchangeably with the term “permanent”.

By “pharmaceutically acceptable” such as in the recitation of a “pharmaceutically acceptable carrier”, a “pharmaceutically acceptable auxiliary” or a “pharmaceutically acceptable salt” is meant herein a material that is not biologically or otherwise undesirable, i.e., the material may be incorporated into a pharmaceutical combination administered to a patient without causing any undesirable biological effects or interacting in a deleterious manner with any of the other components of the combination in which it is contained. “Pharmacologically active”, as in a “pharmacologically active” derivative or metabolite, refers to a derivative or metabolite having the same type of pharmacological activity as the parent compound and approximately equivalent in degree. When the term “pharmaceutically acceptable” is used to refer to a derivative of an active agent, it is to be understood that the compound is pharmacologically active as well, i.e., therapeutically effective for the treatment of radiocontrast media induced nephropathy.

“Carriers” or “pharmaceutically acceptable auxiliary” as used herein refer to conventional pharmaceutically acceptable excipient materials suitable for drug administration and include any such materials known to a person of skill in the art that are nontoxic and do not interact with other components of a pharmaceutical combination or drug delivery system in a deleterious manner.

As used herein, the terms “comprising” and “including” are used herein in their open, non-limiting sense.

The term “prodrug” as used herein, represents derivatives of the compounds disclosed herein that are drug precursors which, following administration to a patient, release or alter the drug in vivo via a chemical or physiological process. As used herein, the term “prodrug” includes metabolic precursors. In particular, prodrugs are derivatives of the compounds disclosed herein in which functional groups carry additional constituents which may be cleaved under physiological conditions in vivo and thereby releasing the active principle of the compound (e.g., a prodrug on being brought to a physiological pH or through an enzyme action is converted to the desired drug form). Prodrugs are bioreversible derivatives of drug molecules used to overcome some barriers to the utility of the parent drug molecule. These barriers include, but are not limited to, solubility, permeability, stability, presystemic metabolism and targeting limitations (Bundgaard, 1985¹⁷). Prodrugs, i.e. compounds that when administered to humans by any known route, are metabolised to compounds having formula I are included within the scope of the present disclosure.

The term “pharmaceutically acceptable salts” refers to salt forms that are pharmacologically acceptable and substantially non-toxic to the subject being administered the compounds described herein. In one embodiment the pharmaceutically acceptable salts is the mesylate salt.

The term “solvates” pertains to the association of suitable organic solvent molecules with molecules or ions of an A1AR. As used herein, the term “solvates” refers both to stable solvates, containing a defined number of solvent molecules per molecule of a compound of formula I, and inclusion complexes, which are less stable and contain a variable number of solvent molecules per molecule of a A1AR.

The term “treatment” as used herein refers to reduction in severity and/or frequency of symptoms, elimination of symptoms and/or underlying cause, prevention of the occurrence of symptoms and/or their underlying cause, and improvement or remediation of damage. Thus, for example, “treatment” of a patient involves prevention of a particular disorder or adverse physiological event in a susceptible individual as well as treatment of a clinically symptomatic individual.

The “increase in serum creatinine level” induced by radiocontrast media can be transient, persistent or irreversible. Reference values for serum creatinine levels (see http://www.mceus.com/renal/renalcreat.html¹⁸) in adult males lies between about 0.8 mg/dl and about 1.4 mg/dl, in adult females between about 0.6 mg/dl and about 1.1 mg/dl and in children between about 0.2 mg/dl and about 1.0 mg/dl. A range of values of between about 25% to about 50% or even higher increase in serum creatinine levels from reference values defines CIN. An “increase in serum creatinine level” as a measurable physiological parameter defines a disease condition well understood by the skilled artisan. An increase of any value within in the range of about 25% to about 70% in serum creatinine levels defines CIN. In a further embodiment, an increase of about 25, about 30, about 35, about 40, about 45, about 50, about 55, about 60, about 65 and about 70%, and every range which lies in any ranges defined by two of the before mentioned values, where the lower limit of said range is defined by the minor value and the upper limit of said range by the higher value, e.g. a range of about 25% to about 30%, about 25% to about 35%, about 30% to about 60%, etc., defines CIN. This definition may in part account for the transient, persistent or irreversible elevation of serum creatinine levels.

The “decrease in renal blood flow” induced by radiocontrast media can be transient, persistent or irreversible. Reference value for blood flow in the kidney is approximately 20% of the cardiac output per minute, thus lies at about 1000 ml/min in a healthy human. A range of values of between about 20% and about 80% or even larger decrease in renal blood flow from reference value defines CIN. A “decrease in renal blood flow” as a measurable hemodynamic parameter defines a disease condition well understood by the skilled artisan. A decrease in value or value range within in the range of about 20% to about 80% in renal blood flow defines CIN. In a further embodiment, a decrease of about 20, about 25, about 30, about 35, about 40, about 45, about 50, about 55, about 60, about 65, about 70, about 75, about 80, about 85 and about 90%, and every value or value range which lies in any ranges defined by two of the before mentioned values, where the lower limit of said range is defined by the minor value and the upper limit of said range by the higher value, e.g. a range of about 25% to about 30%, about 20% to about 35%, about 30%, about 60%, etc., defines CIN. This definition may in part account for the transient, persistent and irreversible decrease in renal blood flow.

The renal blood flow can be measured using MRI (magnetic resonance imaging) techniques to determine renal blood flow and renal vascular resistance as well as PAH (para amino hipuric acid) infusion techniques.

Any of the foregoing A1AR may be administered in the form of a salt, ester, amide, prodrug, active metabolite, analog, solvate or the like, provided that the salt, ester, amide, prodrug, active metabolite, analog, or solvate is pharmaceutically acceptable and pharmacologically active in the present context. Salts, esters, amides, prodrugs, metabolites, analogs, solvates and other derivatives of the active agents may be prepared using procedures known to those skilled in the art of synthetic organic chemistry and described, for example, by J. March (1992¹⁹).

A further embodiment herein is a kit comprising in separate or the same containers in a single package pharmaceutical dosage forms for use in combination, comprising, in one container a pharmaceutical dosage form comprising a first A1AR and in a second container a pharmaceutical dosage form comprising a first RM. The kit form is particularly advantageous but not limited to the case when the separate components must be administered in different dosage forms or are administered at different dosage intervals. The selective adenosine A1 dosage forms may be injectable formulations like solutions and suspensions. The kit may further comprise instructions which will typically be written instructions on a package insert, a label, and/or on other components of the kit, and the intravenous dosage forms are as described herein. Each dosage form may be individually housed. The present kits will also typically include means for packaging the individual kit components, i.e., the dosage forms, the container, and the written instructions for use.

In an embodiment, the therapeutically effective amount of A1AR is administered in a form as set forth above. However, in some cases, a patient may be given each, the therapeutically effective amount of A1AR and the RM, in its own separate dosage form, or a combination of individual “combination” dosage forms containing two or more of the present therapeutically effective A1ARs. When separate dosage forms are used, the A1AR and the RM can be administered at essentially the same time (concurrently), or at separately staggered times (sequentially). Optimum beneficial effects are achieved when the active blood plasma level concentrations of the A1AR agent is maintained during administration of the RM. These optimal beneficial effects can be achieved by application of a loading dose following by one or more maintenance doses. The loading dose will rapidly increase the blood plasma level while the maintenance dose(s) will then serve to retain the desired blood plasma concentration. A form comprising the A1AR and the RM constitutes, however, a further embodiment. Such a dosage form provides convenience and simplicity for the patient, thus increasing the chances for patient compliance. Since two or even more active agents are being used together in combination, the potency of each of the agents and the interactive effects achieved by combining them together must also be taken into account. A consideration of these factors is well within the purview of the ordinarily skilled clinician for the purpose of determining the therapeutically effective or prophylactically effective dosage amounts.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In further embodiments, a straight chain or branched chain alkyl has 30 or fewer carbon atoms in its backbone (e.g., C₁-C₃₀ for straight chain, C₃-C₃₀ for branched chain), and in an embodiment 20 or fewer, e.g. in an embodiment “alkyl” may be C₁-C₆ or in a further embodiment C₁-C₄. Likewise, in an embodiment cycloalkyls have from 4-10 carbon atoms in their ring structure, and in a further embodiment cycloalkyls have from 5-7 carbon atoms in their ring structure, e.g. 5, 6 or 7 carbons in the ring structure. Moreover, the term “optionally substituted alkyl” as used throughout the specification and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above.

An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain a first double or triple bond respectively.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “heterocyclic ring”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “heteroatom” as used herein means an atom of any element other than carbon or hydrogen. In one embodiment the heteroatom is nitrogen.

It will be noted that the structure of some of the compounds disclosed herein include asymmetric carbon atoms. It is to be understood accordingly, that the isomers arising from such asymmetry (e.g., all enantiomers and diastereomers) are included within the scope of the disclosure, unless indicated otherwise. Some isomers can be obtained in substantially pure form by classical separation techniques and by stereochemically controlled synthesis.

The selective adenosine A1 antagonists described herein have low lipophilic properties and therewith high hydrophilic properties resulting in good water solubility. The significantly lower lipophilic properties of the compounds described herein distinguish said compounds over other known selective A1 antagonists; exemplary data is depicted in the table below.

TABLE 1
lipophilic properties of selective adenosine A1 antagonists

| | PGP-factor | Permeability (%) | LogP (ACD v9.05) |
|---|---|---|---|
| substance 1 | 1.5 | 37.4 | 1.6 |
| substance 2 | 10.1 | 27.0 | −1.4 |
| KW3902 | 1.4 | 31.1 | 4.2 |

From the receptor binding and enzyme profiling of substance 1 in a wide range of assays, it was concluded that substance 1 behaved as a selective adenosine A1 receptor ligand with some phosphodiesterase PDE4 inhibiting activity. The displacement of rolipram by substance 1 from phosphodiesterase PDE4 sites correlated with the relative potency of substance 1 to inhibit this enzyme; the calculated pKi of the PDE4 inhibition was 750 nM; the activities on other phosphodiesterases (PDE 1, 2, 3, 5 and 6) were at least 25 fold lower. The phosphodiesterase PDE4 inhibiting activity may be used for titration purposes of patients. The phosphodiesterase PDE4 inhibiting activity of the compounds described herein prevents overdosage of the selective A1 antagonist by alarming the patients with self-evident, non-serious signals like e.g. headache before serious events like e.g. CNS convulsion can occur.

Study Protocol

Study 1

Animal studies were performed in 60 anesthetized rats. Renal hemodynamics were assessed and oxygen tension within the kidney is measured after application of RM. Total blood flow to the kidney was quantified by the transit time method and local hemodynamics by laser-Doppler Flux. In addition, regional oxygen tension of the kidney was assessed and urine collected to determine urine osmolarity, viscosity and diuresis. Using a recently established technique (Wronski, 2003²⁰), it is possibly to assess the TGF response in this setting. The RM significantly reduces renal blood flow and perturbs regional kidney oxygenation. This effect is most likely due to viscous properties, as seen by an increase in urine viscosity. These RM effects on renal hemodynamics (renal blood flow and hypoxia) were alleviated or even reversed by prior administration of the A1AR antagonists.

Two protocols were undertaken. In Protocol 1, fluid restriction took place 24 h before experiments. This led to augmented concentration of RM in the tubular system. Catheters, transit-time flowmeters, laser-Doppler probes and sounds for assessing absolute pO₂ were implanted. Control measurements were recorded, and then the RM was administered. In Protocol 2, measurements were repeated. In the fluid replete animal, urine volume, osmolarity and viscosity were determined. Control measurements were recorded, then, the RM was administered.

Renal blood flow, oxygen tension and regional blood flows and the TGF response in rats was assessed after water restriction took place. Reduced plasma volume is a generally recognized risk factor, since CM is concentrated in the tubules during antidiuresis.

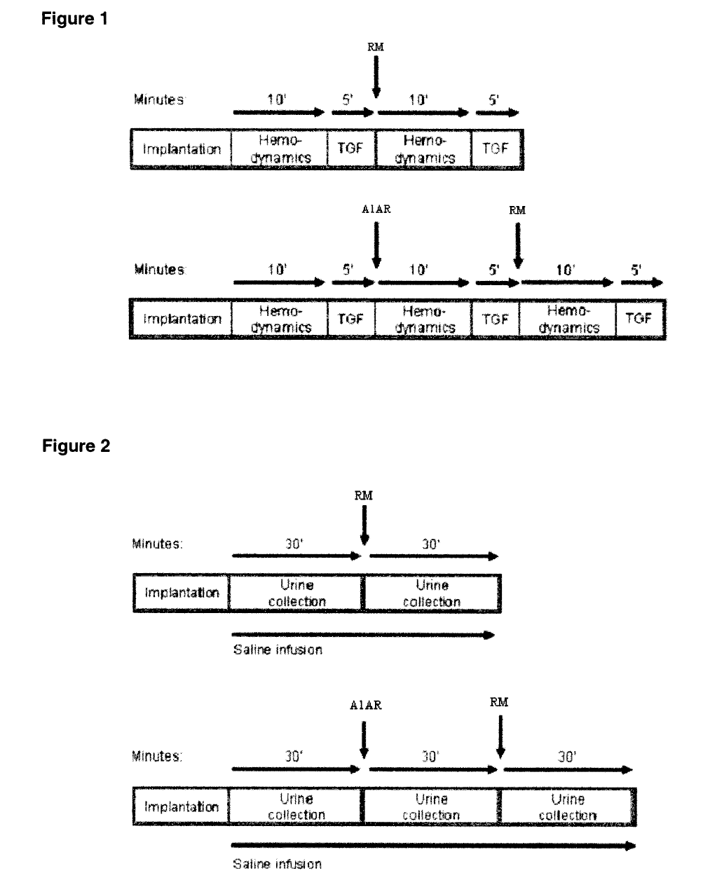

FIG. 1 depicts the protocols. In the top panel, the RM was given after control measurements (N=15). The bottom panel depicts the series where the A1AR is given prior to the RM (N=15).

In order to collect sufficient urine, volume repleted rats were used. Diuresis, urine osmolality and viscosity were assessed for control and the RM (N=15, Error! Reference source not found. top panel), and for control, the A1AR and the A1AR+the RM (N=15, Error! Reference source not found. bottom panel). All experiments were performed on adult, male Wistar rats obtained from the animal facility of the institute. The rats were housed in groups. All animals were randomly distributed between the protocols. The animals were identified by cage number. A standard rat diet (Altromin 1324, Altromin GmbH, D-32791 Lage) served as chow. Feeding and drinking was discontinued approx. 12 hours before the surgery for protocol 1. In protocol 2, drinking was allowed ad libitum. Drinking water was offered ad libitum, except for a time period of 12 h before CM application. Thus, the animals were water deprived. In protocol 2, water was offered ad libitum until immediately before the experiment. Granulated textured wood (Granulat A2, J. Brandenburg, D-49424 Goldenstedt) was used as bedding material for the cages. The cages were changed and cleaned every day between 6:00 and 8:00 a.m. During the acclimatization, the animals were kept in groups of 3-5 animals in MAKROLON cages each (type 4) at a room temperature of 22° C.±3° C. and a relative humidity of 60%±20%. Deviation may be caused, for example, during the cleaning procedures. Anesthesia was introduced and maintained by urethane. Rats were placed on a heated table to maintain body temperature at 37° C. throughout the surgery. The body temperature was controlled during the study. After an incision in the left groin, the femoral artery was carefully prepared and cannulated with a polypropylene catheter (PP 10) to measure the renal perfusion pressure (RPP). Another catheter (PP 50) of the same material was placed into the carotid artery to measure systemic blood pressure (BP) and heart rate (HR). Finally, an inflatable cuff was placed around the abdominal aorta; one above and the other below the origin of the renal arteries. A servo controlled inflation of the proximal cuff allowed it to reduce and maintain renal perfusion pressure at a preset level. Two 500 μm diameter optical fibers (Moore instruments, GB) were implanted into the cortex and the medulla of left kidney, and an ultrasound transit time flowprobe (1RB, Transonic Systems inc, USA) was placed around the renal artery of the same kidney to determine local blood flows (LFC and LFM respectively) and total kidney blood flow (RBF). pO₂ was likewise locally determined. Local blood flow was measured and processed by a laser-Doppler flowmeter (Moore Instruments, GB). The arterial catheter was connected to the calibrated pressure transducer. The inflatable cuff was connected to an extracorporal servo control system and the flow probes were connected via extension cables to the Flowmeters. Oxygen partial pressure sensing probes were positioned in a corresponding manner. After analog to digital conversion all data (BP, RPP, RBF, LFC, LFM, local oxygen tension) were stored on-line in ASCII format by a computer system (IBM compatible AT). After implantation and stabilization, the experiment was started. The test solutions were infused. After 5 min equilibrium, measurements of RBF, local fluxes and local pO₂ was commenced. Then, a 5 min step response was obtained to assess TGF. Urine was collected 35 min to evaluate diuresis, osmolality and viscosity. When required, modifications were made to the protocols. Total and regional RBF and oxygen tension in the renal medulla and cortex were assessed according to prior studies (Flemming, 2000 and 2001²¹) by measuring laser-Doppler-fluxes and direct assessment of pO₂. After the calculation of individual mean values of every parameter, these mean values of every animal were used to calculate group averages and standard errors of each controvintervention group. The latter were used to test differences for statistical significance, in an embodiment levels of less than 0.05 are considered to indicate significance. The test methods used were chosen with respect to the parameters of the underlying data.

Study 2

A study analogous to the one described by Yao (2000¹⁰) with selected variations in the protocol was performed. In contrast to the Yao study, chronic as well as acute experiments were carried out. Indometacine is used in addition to L-name (N-ω-nitro-L-arginine methyl ester) in this study.

### EXPERIMENTAL

1. Effects of Acute Application of Substance 1 on Diuresis and Natriuresis Following Radiocontrast Media (Diatrizoate) in Anesthetized Rats

The radiocontrast media-dependent acute kidney failure was induced using an experimental protocol based on that published by the group of Osswald,²² Male Sprague-Dawley rats of approximately 300 g body weight were acclimatized for at least 1 week before the start of the chronic pretreatment with the nitric oxide synthase inhibitor N-nitro-L-arginine methyl ester (L-NAME for 7-9 weeks at a daily dose of 5 mg/kg). For the experiments, the overnight fasted rats (which had continued free access to drinking water) were anesthetized with Inactin (80 mg/kg, given i.p. as a bolus). Catheters were placed (i) in the trachea, (ii) in one jugular vein (for radiocontrast medium administration, and background saline infusion; see below), (iii) in the other jugular vein for vehicle or sunstance 1 administration, (iv) in the carotid artery for blood sampling, and part of the background saline infusion; see below), and (v) in the bladder for urine collection. The rats were kept on a heated table to maintain their body temperature at 37° C. After collecting urine samples for 60-90 min for baseline measurements, the animals received vehicle or substance 1 as follows: a loading bolus of 0.15, or 1.5 mg substance 1 per kg, or vehicle, in a volume of 1 mukg, was applied intravenously, followed by a continuous intravenous infusion at a rate of 1.5, and 15 pg substance 1 per kg per min, or vehicle, in a volume of 11 μL/kg·min until the end of the experiment. With these dose regimens steady-state plasma levels of substance 1 were 59±23, and 314±36 ng/mL, respectively. Ten minutes after the start of the substance 1 (or vehicle) treatment, diatrizoate (meglumine salt, Urolux, 0.61 g diatrizoate/mL, corresponding to a total iodine content of 290 mg/mL, Sanochemia Diagnostics, Neuss, Germany), prewarmed at body temperature, was infused iv over 3 min at a dose of 2.55 mL/kg, corresponding to 740 mg iodine/kg (the timepoint of contrast medium administration was defined as t₀). A background saline solution was infused from the beginning and maintained at a rate of ˜1.2 mL/h per 100 g until the end of the experiments (0.24 mL/h via the arterial catheter, and 0.96 mL/h via the venous line). This infusion was required to compensate for the volume loss due to the operation, and the subsequent blood sampling, but also to insure the patency of the arterial catheter between the blood sampling timepoints. Urine samples were collected according to the following schedule: baseline (60-90 min preceding the start of substance 1, or vehicle, administration), t₀-30 min (0.5 h timepoint), 30-60 min (1 h timepoint), 60-120 min (2 h timepoint), and 120-180 min (3 h timepoint). Plasma samples were taken at the end of each of the above periods. The measured values of urine volume, and urine levels of Na⁺ were used to calculate the rates of diuresis and natriuresis over the time intervals mentioned above.

As shown in Table 2, substance 1 treatment produced a large and significant increase in urine production in the first 30 min following contrast medium administration, as compared to the vehicle control group; in spite of the fact that the rate of diuresis then returned towards lower values in all groups, a stimulating effect of substance 1 persisted for at least 3 h. Because radiocontrast media are eliminated via the urine, this diuretic effect of substance 1 is likely to strongly promote their elimination and thus to limit their toxicity.

TABLE 2
Effects of substance 1 on diuresis following diatrizoate administration in anesthetized
in rats, Values are expressed in mL per kg body weight per h, and represent means ± SEM
(n = 14 − 24). Statistical significance was evaluated using one-way analysis
of variance followed by a Bonferroni test. n.s.: non significant; *: P < 0.05;
**: P < 0.01; and ***: P < 0.001 vs. vehicle controls.

| Time after | Vehicle | Substance 1 | | Substance 1 | |
|---|---|---|---|---|---|
| CM | control | Low dose | P vs. vehicle | high dose | P vs. vehicle |
| 0.5 h | 17.10 ± 1.42 | 27.31 ± 2.36 | *** | 28.01 ± 2.02 | *** |
| 1 h | 3.76 ± 0.31 | 3.87 ± 0.40 | n.s. | 4.97 ± 0.30 | n.s. |
| 2 h | 1.96 ± 0.15 | 3.34 ± 0.42 | ** | 3.13 ± 0.27 | ** |
| 3 h | 2.14 ± 0.40 | 3.54 ± 0.50 | n.s. | 3.11 ± 0.36 | n.s. |

Likewise, substance 1 caused a pronounced and sustained increase in sodium excretion over values seen in the vehicle control group (Table 3). Chloride excretion was stimulated by substance 1 in a similar way, whereas potassium excretion was not relevantly affected by the compound throughout eh experiment (not shown).

TABLE 3
Effects of substance 1 on sodium excretion following diatrizoate administration
in anesthetized in rats, Values are expressed in μmol per kg body weight
per h, and represent means ± SEM (n = 14 − 24). Statistical
significance was evaluated using one-way analysis of variance followed by
a Bonferroni test. n.s.: non significant; *: P < 0.05; **: P < 0.01;
and ***: P < 0.001 vs. vehicle controls.

| Time after | Vehicle | Substance 1 | | Substance 1 | |
|---|---|---|---|---|---|
| CM | control | Low dose | P vs. vehicle | high dose | P vs. vehicle |
| 0.5 h | 1332 ± 184 | 3039 ± 354 | *** | 3301 ± 306 | *** |
| 1 h | 185 ± 39 | 338 ± 81 | n.s. | 520 ± 77 | ** |
| 2 h | 153 ± 40 | 613 ± 121 | ** | 591 ± 88 | ** |
| 3 h | 329 ± 72 | 842 ± 121 | ** | 830 ± 91 | ** |

2. Effects of Acute Application of Substance 1 on Renal Blood Flow and Oxygenation Following Radiocontrast Media (Iodixanol) in Anesthetized Rats

The experimental protocol was based on a previously published methodology. The experiments were performed using adult male 3-4 months old Wistar rats. Body weight ranged from 250 to 400 g. The rats received a standard chow diet. Feeding and drinking was discontinued approx. 12 hours before the surgery. The animals were anesthetized by intraperitoneal injection of urethane solution (2% in water; 6 ml per kg), and placed on a heated table to maintain body temperature at 37° C. throughout surgery and subsequent experiments. After an incision in the left groin the femoral artery was carefully prepared and cannulated to measure the mean arterial blood pressure. Another catheter was placed into the carotid artery for administration of contrast medium. Finally, an inflatable cuff was placed around the abdominal aorta above the origin of the renal arteries. A servo controlled inflation of the cuff allowed for the reduction and maintenance of the renal perfusion pressure at a preset levels. Two 500 μm diameter optical fibers were implanted into the cortex and the outer medulla of left kidney to determine local laser-Doppler fluxes and an ultrasound transit time flowprobe was placed around the renal artery of the same kidney to determine total kidney blood flow (RBF). Renal oxygen levels (oxygen partial pressure=pO₂) was likewise determined locally (cortical and medullary pO₂, respectively; OxyLite, Oxford Optronics). After implantation and stabilization, the experiment was started by measuring hemodynamic and oxygenation parameters under baseline conditions. Vehicle or substance 1 (5 mg/kg as an intravenous bolus) were then administered. New measurements were performed, and 30 min after vehicle or substance 1 administration, lodixanol (Visipaque 320; 1.5 mL i.a.; Amersham Buchier, Braunschweig, Germany), or vehicle was applied. After another 20 min, measurements were repeated (over a period of 20 min, shown in figures below). The experimental groups were thus: 1. Vehicle+Vehicle (‘control’), Vehicle+Visipaque, and sunstance 1+Visipaque.

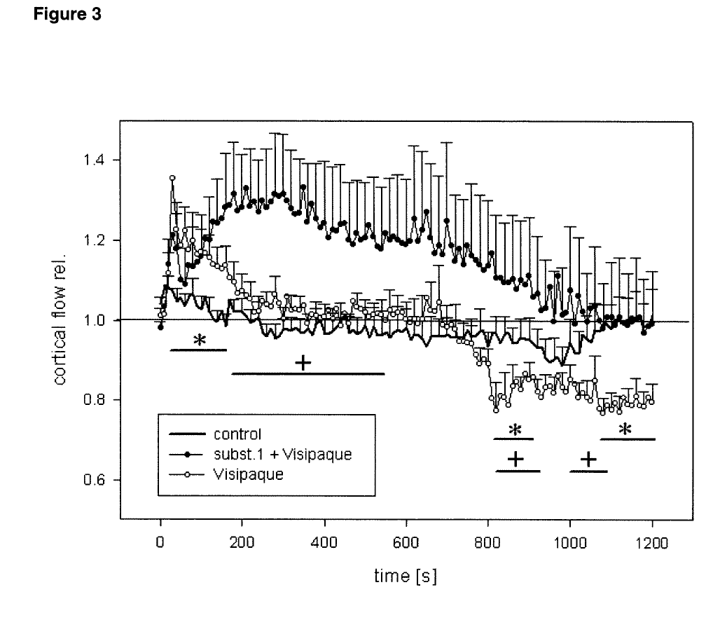

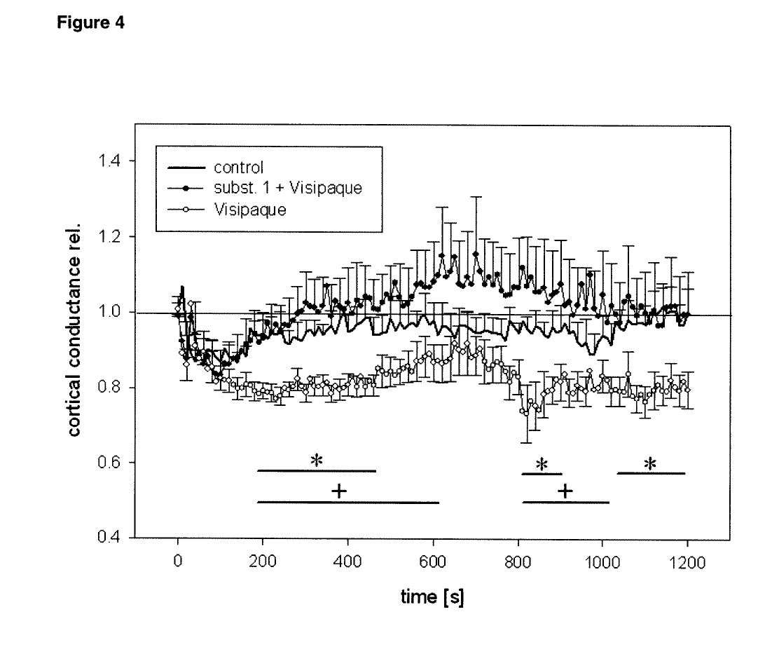

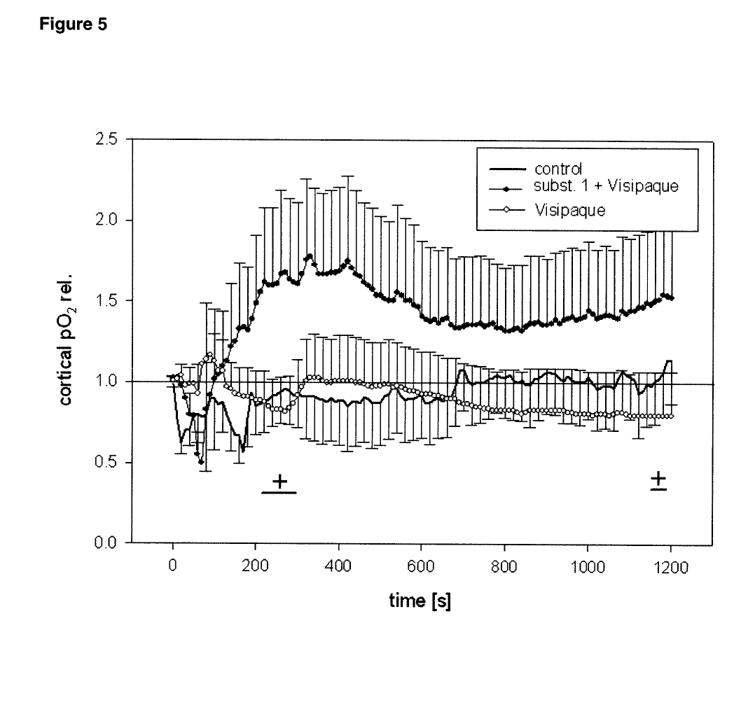

Substance 1 did not modify hemodynamic parameters (arterial blood pressure, RBF) before the Visipaque challenge (not shown). Following Visipaque administration, a strong, transient increase in mean arterial blood pressure was observed (by ˜35 mm Hg), which lasted for approximately 10 min, and was partially prevented by sunstance 1 (not shown). In the Vehicle+Visipaque group, renal cortical blood flow showed a short initial increase which was followed by a progressive and significant decrease, as compared to the vehicle control group (FIG. 3). In contrast, in the presence of substance 1, cortical blood flow displayed a sustained and significant increase, blood flow remaining significantly elevated until the end of the measurement period as compared to the Vehicle+Visipaque group (FIG. 3). Cortical vascular conductance was rapidly and stably lowered by Visipaque, whereas substance 1 maintained this parameter at levels seen in the vehicle control group (FIG. 4). Similarly, medullary blood flow transiently rose (for ˜3 min) after Visipaque injection, and then fell below control levels; concomitantly, medullary vascular conductance was rapidly and stably depressed by Visipaque; substance 1 treatment only partially (but significantly) prevented these effects (not shown). Finally, substance 1 caused a significant increase in cortical pO₂ which persisted until the end of the experiment (FIG. 5).

Overall, these observations show that substance 1 improves renal hemodynamics and oxygenation, thus at least partially antagonizing the potentially deleterious effects of the radiocontrast medium iodixanol.

U.S. patent application Ser. No. ______ filed on Jun. 19, 2007 and claiming the benefit of U.S. Provisional Patent Application Nos. 60/805,168 and 60/805,173 filed on Jun. 19, 2006 and U.S. Provisional Application No. 60/871,062 filed on Dec. 20, 2006 is hereby incorporated by reference in its entirety.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of any aspect of the present disclosure.

Alternative embodiments of the claimed disclosure are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or limitation at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

### CITED DOCUMENTS

¹ Andrew E, Berg K J, “Nephrotoxic effects of X-ray contrast media”, J Toxicol Clin Toxicol. 2004; 42(3):325-32
² http://www.fpnotebook.com/REN70.htm and http://www.fpnotebook.com/REN38.htm (15 Jun. 2006)
³ Ueda J, Nygren A, Hansell P, Ulfendahl H R. “Effect of intravenous contrast media on proximal and distal tubular hydrostatic pressure in the rat kidney”. Acta Radiol 1993; 34(1):83-87
⁴ Olivera A, Lamas S, Rodriguez-Puyol D, Lopez-Novoa J M. “Adenosine induces mesangial cell contraction by an A1-type receptor”. Kidney Int 1989; 35(6):1300-1305
⁵ Porter, “Contrast-associated nephropathy” Am. J. Cardiol., (1989), 64(9), 22E-26E
⁶ Nikolsky E, Aymong E D, Dangas G, Mehran R. “Radiocontrast nephropathy: identifying the high-risk patient and the implications of exacerbating renal function”, Rev Cardiovasc Med 2003; 4 Suppl 1:S7-S14
⁷a) Shammas et al., “Aminophylline does protect against radiocontrast nephropathy in patients undergoing percutaneous angiographic procedures”, J Invasive Cardiol (2001), 13(11), 738-40; b) Welch et al. “Adenosine A1 receptor antagonists in the kidney: effects in fluid-retaining disorders”, Curr Opin Pharmacol (2002), 2(2), 165-70; c) Huber et al. “Effect of theophylline on contrast material-nephropathy in patients with chronic renal insufficiency: controlled, randomized, double-blinded study”, Radiology (2002), 223(3), 772-9
⁸ Erley et. al., “Adenosineantagonist theophylline prevents the reduction of glomerularfiltration rate after contrast media application”, Kidney Int. (1994), 45, 1425-31
⁹ K. Akawara et al., “Role of adenosine in the renal responses to contrast medium”, Kidney Int. (1996), 49(5), 1199-206
¹⁰ K. Yao et al., “The selective adenosine A1 receptor antagonist KW-3902 prevents radiocontrast media-induced nephropathy in rats with chronic nitric oxide deficiency”, Europ J of Pharmacology (2001) 414, 99-104
¹¹ Greiner, Dissertation “Prophylaxis of Contrast Induced Nephropathy with Theophylline and Acetylcysteine in ICU-Patients”, TU München, 19 Oct. 2005
¹² H. Thomas Lee, Michael Jan, Soo Chan Bae, Jin Deok Joo, Farida R. Goubaeva, Jay Yang, and Mihwa Kim, “A₁ adenosine receptor knockout mice are protected against acute radiocontrast nephropathy in vivo”, Am J Physiol Renal Physiol 290: F1367-F1375, 2006
¹³ EP 1 386 609, filed by CV Therapeutics
¹⁴ WO 99/31101, filed by Univ. South Florida
¹⁵ WO 99/62518, WO 01/39777, WO 02/057267, all filed by Osi Pharmaceuticals and WO 2004/094428 filed by Solvay Pharmaceuticals
¹⁶ EP 0 022 744 filed by Schering, EP 0 023 992 filed by Bracco Industria Chimica, EP 0 026 281 filed by Bracco Industria Chimica, EP 0 033 426 filed by Univ. California, EP 0 108 638 filed by Nyegaard, EP 0 317 492 filed by Schering, WO 87/00757 filed by Cook Imaging Corporation, WO 89/08101 filed by Mallinckrodt, U.S. Pat. No. 2,776,241 filed by Schering, U.S. Pat. No. 3,290,366 filed by Mallinckrodt, U.S. Pat. No. 3,360,436 filed by Eprova, U.S. Pat. No. 5,349,085 filed by Nycomed, GB 1 321 591 filed by Nyegaard, DE 2 547 789 filed by Savag, DE 2 726 196 filed by Nyegaard, DE 2 909 439 filed by Schering
¹⁷ Bundgaard, H. (editor), “Design of Prodrugs”, Elsevier, 1985
¹⁸ http://www.mceus.com/renal/renalcreat.html (15 Jun. 2006)
¹⁹ Advanced Organic Chemistry: Reactions, Mechanisms and Structure, 4th Edition, New York:Wiley-Interscience, 1992
²⁰ Wronski T, Seeliger E, Persson P B, Former C, Fichtner C, Scheller J et al. The step response: a method to characterize mechanisms of renal blood flow autoregulation. Am J Physiol Renal Physiol 2003; 285(4):F758-F764
²¹ a) Flemming B, Arenz N, Seeliger E, Wronski T, Steer K, Persson P B. Time-dependent autoregulation of renal blood flow in conscious rats. J Am Soc Nephrol 2001; 12(11):2253-2262 and b) Flemming B, Seeliger E, Wronski T, Steer K, Arenz N, Persson P B. Oxygen and renal hemodynamics in the conscious rat. J Am Soc Nephrol 2000; 11(1):18-24
²² Erley C M, Heyne N, Burgert K, Langanke J, Risler T, & Osswald H (1997): Prevention of Radiocontrast-Induced Nephropathy by Adenosine Antagonists in Rats with Chronic Nitric Oxide Deficiency. J. Am. Soc. Nephrol., 8:1125-1132

We claim:

1. A method of preventing radiocontrast media induced nephropathy in mammals or humans comprising administering a therapeutically effective amount of a first selective adenosine A1 antagonist.

2. The method of claim 1 wherein the first selective adenosine A1 receptor antagonist is administered intravenously in a loading dose followed by subsequent administration as a maintenance dose, wherein the loading dose is administered between about five minutes and about twenty-five minutes prior to the administration of a first radiocontrast media and wherein the maintenance dose is administered over a period of less than about 48 hours subsequent to administration of the loading dose.

3. The method of claim 2 wherein the maintenance dose is administered such that the plasma level of the first selective A1 adenosine antagonist is maintained between about 10 ng/ml and about 500 ng/ml.

4. The method of claim 1 wherein the first adenosine A1 receptor antagonist is administered orally prior to the administration of a first radiocontrast media.

5. The method of claim 1 wherein the first radiocontrast media is not administered until the plasma concentration level of the first adenosine A1 receptor antagonist has reached a concentration of between about 10 ng/ml and about 500 ng/ml.

6. The method of claim 1 wherein the first selective adenosine A1 receptor antagonist is selected from pyrrolo[2,3d]pyrimidine derivatives of formula I 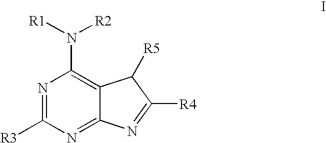
wherein
i) R1 and R2 are each independently selected from a hydrogen atom, an optionally substituted alkyl, optionally substituted aryl, or optionally substituted alkylaryl moiety or together form an optionally substituted heterocyclic ring;
ii) R3 is selected from a hydrogen atom or an optionally substituted alkyl, optionally substituted aryl, or optionally substituted alkylaryl moiety;
iii) R4 and R5 are each independently selected from a halogen atom, a hydrogen atom or an optionally substituted alkyl, optionally substituted aryl, or optionally substituted alkylaryl moiety, or R4 and R5 together form an optionally substituted heterocyclic or optionally substituted carbocyclic ring;
and pharmaceutically acceptable salts of the foregoing, pharmaceutically acceptable prodrugs of the foregoing, and pharmaceutically acceptable solvates of the foregoing.

7. The method of claim 6 wherein the first selective adenosine A1 receptor antagonist is selected from pyrrolo[2,3d]pyrimidine derivatives of formula I, wherein
i) R1 and R2 are each independently selected from a hydrogen atom, an optionally substituted alkyl or together form an optionally substituted heterocyclic ring;
ii) R3 is a hydrogen atom or an optionally substituted aryl,
iii) R4 and R5 are each independently selected from a halogen atom or a hydrogen atom;
and pharmaceutically acceptable salts of the foregoing, pharmaceutically acceptable prodrugs of the foregoing, and pharmaceutically acceptable solvates of the foregoing.

8. The method of claim 7 wherein the selective adenosine A1 receptor antagonist is selected from pyrrolo[2,3d]pyrimidine derivatives of formula I, wherein
i) R1 is a hydrogen and R2 is an optionally substituted cyclohexyl ring, or R1 and R2 together form an optionally substituted pyrrolidine ring;
ii) R3 is a phenyl ring,
iii) R4 and R5 are each a hydrogen atom;
and pharmaceutically acceptable salts of the foregoing, pharmaceutically acceptable prodrugs of the foregoing, and pharmaceutically acceptable solvates of the foregoing.

9. The method of claim 8 wherein the first selective adenosine A1 receptor antagonist is selected from the group consisting of: 4-[(2-phenyl-7H-pyrrolo[2,3-o]pyrimidin-4-yl)amino]-trans-cyclohexanol methanesulfonate, (4S)-4-hydroxy-1-(2-phenyl-7H-pyrrolo[2,3-d]pyrimidin-4-yl)-L-prolinamide methanesulfonate, pharmaceutically acceptable prodrugs of the foregoing, and pharmaceutically acceptable solvates of the foregoing.

10. The method of claim 9 wherein the first selective adenosine A1 receptor antagonist is 4-[(2-phenyl-7H-pyrrolo[2,3-d]pyrimidin-4-yl)amino]-trans-cyclohexanol methanesulfonate, pharmaceutically acceptable prodrugs of the foregoing, and pharmaceutically acceptable solvates of the foregoing.

11. The method of claim 9 wherein the first selective adenosine A1 receptor antagonist is (4S)-4-hydroxy-1-(2-phenyl-7H-pyrrolo[2,3-d]pyrimidin-4-yl)-L-prolinamide methanesulfonate, pharmaceutically acceptable prodrugs of the foregoing, and pharmaceutically acceptable solvates of the foregoing.

12. The method of claim 2 wherein the first radiocontrast media is an iodinated or gadolinium-based radiocontrast media selected from the group consisting of bunaiod, biligram, bilimiro, bilopaque, cholimil, ethiodol, diatrast, dionosil, falignost, gadobutrol, gadodiamide, gadopentetate dimeglumine, gastrografin, hexabrix, hippodin, mangafodipir, amidotrizoate, ethiodized oil, imagopaque, iodamide, iodipamide, iodixanol, iodophene, iophendylate, iomeron, iomeprol, iopamidol, iopanoic acid, iopiperidol, iophendylate, iopromide, iopydol, iosimenol, iothalamic acid, iotrolan, ioversol, ioxilan, ioxaglic acid, isopaque, ipodate, meglumine iothalamate, meglumine acetrizoate, meglumine diatrizoate, metrizamide, myelotrast, omnipaque, osbil, optiray, optojod, opacoron, perflutren, phenobutiodil, phentetiothalein sodium, priodax, propyliodone, skiodan, sodium iodomethamate, sodium diatrizoate, telepaque, teridax, tetrabrom, thorotrast, triognost, 1,3,5-Tri-n-hexyl-2,4,6-triiodobenzene, tyropanoate, visipaque or xenetix, pharmaceutically acceptable salts of any of the foregoing, prodrugs of any of the foregoing, and solvates of any of the foregoing.

13. A method of preventing a radiocontrast media induced increase in serum creatinine levels in mammals or humans comprising administering a therapeutically effective amount of a first selective adenosine A1 antagonist.

14. The method of claim 13 wherein the first selective adenosine A1 receptor antagonist is administered intravenously in a loading dose followed by subsequent administration as a maintenance dose, wherein the loading dose is administered between about five minutes and about twenty-five minutes prior to the administration of a first radiocontrast media and wherein the maintenance dose is administered over a period of less than about 48 hours subsequent to administration of the loading dose.

15. The method of claim 14 wherein the maintenance dose is administered such that the plasma level of the first selective A1 adenosine antagonist is maintained between about 10 ng/ml and about 500 ng/ml.

16. The method of claim 13 wherein the first adenosine A1 receptor antagonist is administered orally prior to the administration of a first radiocontrast media.

17. The method of claim 13 wherein the first radiocontrast media is not administered until the plasma concentration level of the first adenosine A1 receptor antagonist has reached a concentration of between about 10 ng/ml and about 500 ng/ml.

18. The method of claim 13 wherein the first selective adenosine A1 receptor antagonist is selected from pyrrolo[2,3d]pyrimidine derivatives of formula I 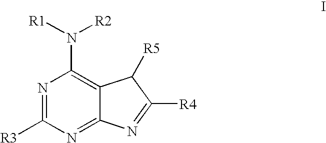
wherein
i) R1 and R2 are each independently selected from a hydrogen atom, an optionally substituted alkyl, optionally substituted aryl, or optionally substituted alkylaryl moiety or together form an optionally substituted heterocyclic ring;
ii) R3 is selected from a hydrogen atom or an optionally substituted alkyl, optionally substituted aryl, or optionally substituted alkylaryl moiety;
iii) R4 and R5 are each independently selected from a halogen atom, a hydrogen atom or an optionally substituted alkyl, optionally substituted aryl, or optionally substituted alkylaryl moiety, or R4 and R5 together form an optionally substituted heterocyclic or optionally substituted carbocyclic ring;
and pharmaceutically acceptable salts of the foregoing, pharmaceutically acceptable prodrugs of the foregoing, and pharmaceutically acceptable solvates of the foregoing.

19. The method of claim 18 wherein the first selective adenosine A1 receptor antagonist is selected from pyrrolo[2,3d]pyrimidine derivatives of formula I, wherein
i) R1 and R2 are each independently selected from a hydrogen atom, an optionally substituted alkyl or together form an optionally substituted heterocyclic ring;
ii) R3 is a hydrogen atom or an optionally substituted aryl,
iii) R4 and R5 are each independently selected from a halogen atom or a hydrogen atom;
and pharmaceutically acceptable salts of the foregoing, pharmaceutically acceptable prodrugs of the foregoing, and pharmaceutically acceptable solvates of the foregoing.

20. The method of claim 19 wherein the selective adenosine A1 receptor antagonist is selected from pyrrolo[2,3d]pyrimidine derivatives of formula I, wherein
i) R1 is a hydrogen and R2 is an optionally substituted cyclohexyl ring, or R1 and
R2 together form an optionally substituted pyrrolidine ring;
ii) R3 is a phenyl ring,
iii) R4 and R5 are each a hydrogen atom;
and pharmaceutically acceptable salts of the foregoing, pharmaceutically acceptable prodrugs of the foregoing, and pharmaceutically acceptable solvates of the foregoing.

21. The method of claim 20 wherein the first selective adenosine A1 receptor antagonist is selected from the group consisting of: 4-[(2-phenyl-7H-pyrrolo[2,3-d]pyrimidin-4-yl)amino]-trans-cyclohexanol methanesulfonate, (4S)-4-hydroxy-1-(2-phenyl-7H-pyrrolo[2,3-d]pyrimidin-4-yl)-L-prolinamide methanesulfonate, pharmaceutically acceptable prodrugs of the foregoing, and pharmaceutically acceptable solvates of the foregoing.

22. The method of claim 21 wherein the first selective adenosine A1 receptor antagonist is 4-[(2-phenyl-7H-pyrrolo[2,3-pyrimidin-4-yl)amino]-trans-cyclohexanol methanesulfonate, pharmaceutically acceptable prodrugs of the foregoing, and pharmaceutically acceptable solvates of the foregoing.

23. The method of claim 21 wherein the first selective adenosine A1 receptor antagonist is (4S)-4-hydroxy-1-(2-phenyl-7H-pyrrolo[2,3-o]pyrimidin-4-yl)-L-prolinamide methanesulfonate, pharmaceutically acceptable prodrugs of the foregoing, and pharmaceutically acceptable solvates of the foregoing.

24. The method of claim 14 wherein the first radiocontrast media is an iodinated or gadolinium-based radiocontrast media selected from the group consisting of bunaiod, biligram, bilimiro, bilopaque, cholimil, ethiodol, diatrast, dionosil, falignost, gadobutrol, gadodiamide, gadopentetate dimeglumine, gastrografin, hexabrix, hippodin, mangafodipir, amidotrizoate, ethiodized oil, imagopaque, iodamide, iodipamide, iodixanol, iodophene, iophendylate, iomeron, iomeprol, iopamidol, iopanoic acid, iopiperidol, iophendylate, iopromide, iopydol, iosimenol, iothalamic acid, iotrolan, ioversol, ioxilan, ioxaglic acid, isopaque, ipodate, meglumine iothalamate, meglumine acetrizoate, meglumine diatrizoate, metrizamide, myelotrast, omnipaque, osbil, optiray, optojod, opacoron, perflutren, phenobutiodil, phentetiothalein sodium, priodax, propyliodone, skiodan, sodium iodomethamate, sodium diatrizoate, telepaque, teridax, tetrabrom, thorotrast, triognost, 1,3,5-Tri-n-hexyl-2,4,6-triiodobenzene, tyropanoate, visipaque or xenetix, pharmaceutically acceptable salts of any of the foregoing, prodrugs of any of the foregoing, and solvates of any of the foregoing.

25. A method of preventing a radiocontrast media induced decrease in renal blood flow in mammals or humans comprising administering a therapeutically effective amount of a first selective adenosine A1 antagonist.

26. The method of claim 25 wherein the first selective adenosine A1 receptor antagonist is administered intravenously in a loading dose followed by subsequent administration as a maintenance dose, wherein the loading dose is administered between about five minutes and about twenty-five minutes prior to the administration of a first radiocontrast media and wherein the maintenance dose is administered over a period of less than about 48 hours subsequent to administration of the loading dose.

27. The method of claim 26 wherein the maintenance dose is administered such that the plasma level of the first selective A1 adenosine antagonist is maintained between about 10 ng/ml and about 500 ng/ml.

28. The method of claim 25 wherein the first adenosine A1 receptor antagonist is administered orally prior to the administration of a first radiocontrast media.

29. The method of claim 25 wherein the first radiocontrast media is not administered until the plasma concentration level of the first adenosine A1 receptor antagonist has reached a concentration of between about 10 ng/ml and about 500 ng/ml.

30. The method of claim 25 wherein the first selective adenosine A1 receptor antagonist is selected from pyrrolo[2,3d]pyrimidine derivatives of formula I 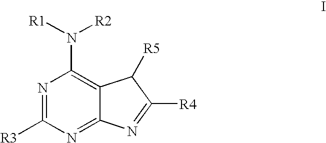
wherein
i) R1 and R2 are each independently selected from a hydrogen atom, an optionally substituted alkyl, optionally substituted aryl, or optionally substituted alkylaryl moiety or together form an optionally substituted heterocyclic ring;
ii) R3 is selected from a hydrogen atom or an optionally substituted alkyl, optionally substituted aryl, or optionally substituted alkylaryl moiety;
iii) R4 and R5 are each independently selected from a halogen atom, a hydrogen atom or an optionally substituted alkyl, optionally substituted aryl, or optionally substituted alkylaryl moiety, or R4 and R5 together form an optionally substituted heterocyclic or optionally substituted carbocyclic ring;
and pharmaceutically acceptable salts of the foregoing, pharmaceutically acceptable prodrugs of the foregoing, and pharmaceutically acceptable solvates of the foregoing.

31. The method of claim 30 wherein the first selective adenosine A1 receptor antagonist is selected from pyrrolo[2,3d]pyrimidine derivatives of formula I, wherein
i) R1 and R2 are each independently selected from a hydrogen atom, an optionally substituted alkyl or together form an optionally substituted heterocyclic ring;
ii) R3 is a hydrogen atom or an optionally substituted aryl,
iii) R4 and R5 are each independently selected from a halogen atom or a hydrogen atom;
and pharmaceutically acceptable salts of the foregoing, pharmaceutically acceptable prodrugs of the foregoing, and pharmaceutically acceptable solvates of the foregoing.

32. The method of claim 31 wherein the selective adenosine A1 receptor antagonist is selected from pyrrolo[2,3d]pyrimidine derivatives of formula I, wherein
i) R1 is a hydrogen and R2 is an optionally substituted cyclohexyl ring, or R1 and
R2 together form an optionally substituted pyrrolidine ring;
ii) R3 is a phenyl ring,
iii) R4 and R5 are each a hydrogen atom;
and pharmaceutically acceptable salts of the foregoing, pharmaceutically acceptable prodrugs of the foregoing, and pharmaceutically acceptable solvates of the foregoing.

33. The method of claim 32 wherein the first selective adenosine A1 receptor antagonist is selected from the group consisting of: 4-[(2-phenyl-7H-pyrrolo[2,3-d]pyrimidin-4-yl)amino]-trans-cyclohexanol methanesulfonate, (4S)-4-hydroxy-1-(2-phenyl-7H-pyrrolo[2,3-d]pyrimidin-4-yl)-L-prolinamide methanesulfonate, pharmaceutically acceptable prodrugs of the foregoing, and pharmaceutically acceptable solvates of the foregoing.

34. The method of claim 33 wherein the first selective adenosine A1 receptor antagonist is 4-[(2-phenyl-7H-pyrrolo[2,3-o]pyrimidin-4-yl)amino]-trans-cyclohexanol methanesulfonate, pharmaceutically acceptable prodrugs of the foregoing, and pharmaceutically acceptable solvates of the foregoing.

35. The method of claim 33 wherein the first selective adenosine A1 receptor antagonist is (4S)-4-hydroxy-1-(2-phenyl-7H-pyrrolo[2,3-d]pyrimidin-4-yl)-L-prolinamide methanesulfonate, pharmaceutically acceptable prodrugs of the foregoing, and pharmaceutically acceptable solvates of the foregoing.

36. The method of claim 26 wherein the first radiocontrast media is an iodinated or gadolinium-based radiocontrast media selected from the group consisting of bunaiod, biligram, bilimiro, bilopaque, cholimil, ethiodol, diatrast, dionosil, falignost, gadobutrol, gadodiamide, gadopentetate dimeglumine, gastrografin, hexabrix, hippodin, mangafodipir, amidotrizoate, ethiodized oil, imagopaque, iodamide, iodipamide, iodixanol, iodophene, iophendylate, iomeron, iomeprol, iopamidol, iopanoic acid, iopiperidol, iophendylate, iopromide, iopydol, iosimenol, iothalamic acid, iotrolan, ioversol, ioxilan, ioxaglic acid, isopaque, ipodate, meglumine iothalamate, meglumine acetrizoate, meglumine diatrizoate, metrizamide, myelotrast, omnipaque, osbil, optiray, optojod, opacoron, perflutren, phenobutiodil, phentetiothalein sodium, priodax, propyliodone, skiodan, sodium iodomethamate, sodium diatrizoate, telepaque, teridax, tetrabrom, thorotrast, triognost, 1,3,5-Tri-n-hexyl-2,4,6-triiodobenzene, tyropanoate, visipaque or xenetix, pharmaceutically acceptable salts of any of the foregoing, prodrugs of any of the foregoing, and solvates of any of the foregoing.

37. A method of preventing or reducing the need of dialysis in a human or mammalian patient receiving a first radiocontrast media comprising administering a therapeutically effective amount of a first selective adenosine A1 antagonist.

38. The method of claim 37 wherein the first selective adenosine A1 receptor antagonist is administered intravenously in a loading dose followed by subsequent administration as a maintenance dose, wherein the loading dose is administered between about five minutes and about twenty-five minutes prior to the administration of a first radiocontrast media and wherein the maintenance dose is administered over a period of less than about 48 hours subsequent to administration of the loading dose.

39. The method of claim 38 wherein the maintenance dose is administered such that the plasma level of the first selective A1 adenosine antagonist is maintained between about 10 ng/ml and about 500 ng/ml.

40. The method of claim 37 wherein the first adenosine A1 receptor antagonist is administered orally prior to the administration of a first radiocontrast media.

41. The method of claim 37 wherein the first radiocontrast media is not administered until the plasma concentration level of the first adenosine A1 receptor antagonist has reached a concentration of between about 10 ng/ml and about 500 ng/ml.

42. The method of claim 37 wherein the first selective adenosine A1 receptor antagonist is selected from pyrrolo[2,3d]pyrimidine derivatives of formula I 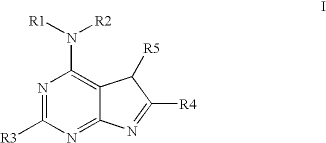
wherein
i) R1 and R2 are each independently selected from a hydrogen atom, an optionally substituted alkyl, optionally substituted aryl, or optionally substituted alkylaryl moiety or together form an optionally substituted heterocyclic ring;
ii) R3 is selected from a hydrogen atom or an optionally substituted alkyl, optionally substituted aryl, or optionally substituted alkylaryl moiety;
iii) R4 and R5 are each independently selected from a halogen atom, a hydrogen atom or an optionally substituted alkyl, optionally substituted aryl, or optionally substituted alkylaryl moiety, or R4 and R5 together form an optionally substituted heterocyclic or optionally substituted carbocyclic ring;
and pharmaceutically acceptable salts of the foregoing, pharmaceutically acceptable prodrugs of the foregoing, and pharmaceutically acceptable solvates of the foregoing.

43. The method of claim 42 wherein the first selective adenosine A1 receptor antagonist is selected from pyrrolo[2,3d]pyrimidine derivatives of formula I, wherein
i) R1 and R2 are each independently selected from a hydrogen atom, an optionally substituted alkyl or together form an optionally substituted heterocyclic ring;
ii) R3 is a hydrogen atom or an optionally substituted aryl,
iii) R4 and R5 are each independently selected from a halogen atom or a hydrogen atom;
and pharmaceutically acceptable salts of the foregoing, pharmaceutically acceptable prodrugs of the foregoing, and pharmaceutically acceptable solvates of the foregoing.

44. The method of claim 43 wherein the selective adenosine A1 receptor antagonist is selected from pyrrolo[2,3d]pyrimidine derivatives of formula I, wherein
i) R1 is a hydrogen and R2 is an optionally substituted cyclohexyl ring, or R1 and
R2 together form an optionally substituted pyrrolidine ring;
ii) R3 is a phenyl ring,
iii) R4 and R5 are each a hydrogen atom;
and pharmaceutically acceptable salts of the foregoing, pharmaceutically acceptable prodrugs of the foregoing, and pharmaceutically acceptable solvates of the foregoing.

45. The method of claim 44 wherein the first selective adenosine A1 receptor antagonist is selected from the group consisting of: 4-[(2-phenyl-7H-pyrrolo[2,3-o]pyrimidin-4-yl)amino]-trans-cyclohexanol methanesulfonate, (4S)-4-hydroxy-1-(2-phenyl-7H-pyrrolo[2,3-d]pyrimidin-4-yl)-L-prolinamide methanesulfonate, pharmaceutically acceptable prodrugs of the foregoing, and pharmaceutically acceptable solvates of the foregoing.

46. The method of claim 45 wherein the first selective adenosine A1 receptor antagonist is 4-[(2-phenyl-7H-pyrrolo[2,3-a]pyrimidin-4-yl)amino]-trans-cyclohexanol methanesulfonate, pharmaceutically acceptable prodrugs of the foregoing, and pharmaceutically acceptable solvates of the foregoing.

47. The method of claim 45 wherein the first selective adenosine A1 receptor antagonist is (4S)-4-hydroxy-1-(2-phenyl-7H-pyrrolo[2,3-d]pyrimidin-4-yl)-L-prolinamide methanesulfonate, pharmaceutically acceptable prodrugs of the foregoing, and pharmaceutically acceptable solvates of the foregoing.

48. The method of claim 38 wherein the first radiocontrast media is an iodinated or gadolinium-based radiocontrast media selected from the group consisting of bunaiod, biligram, bilimiro, bilopaque, cholimil, ethiodol, diatrast, dionosil, falignost, gadobutrol, gadodiamide, gadopentetate dimeglumine, gastrografin, hexabrix, hippodin, mangafodipir, amidotrizoate, ethiodized oil, imagopaque, iodamide, iodipamide, iodixanol, iodophene, iophendylate, iomeron, iomeprol, iopamidol, iopanoic acid, iopiperidol, iophendylate, iopromide, iopydol, iosimenol, iothalamic acid, iotrolan, ioversol, ioxilan, ioxaglic acid, isopaque, ipodate, meglumine iothalamate, meglumine acetrizoate, meglumine diatrizoate, metrizamide, myelotrast, omnipaque, osbil, optiray, optojod, opacoron, perflutren, phenobutiodil, phentetiothalein sodium, priodax, propyliodone, skiodan, sodium iodomethamate, sodium diatrizoate, telepaque, teridax, tetrabrom, thorotrast, triognost, 1,3,5-Tri-n-hexyl-2,4,6-triiodobenzene, tyropanoate, visipaque or xenetix, pharmaceutically acceptable salts of any of the foregoing, prodrugs of any of the foregoing, and solvates of any of the foregoing.

49. A pharmaceutical combination comprising
i) a therapeutically effective amount of a first selective adenosine A1 antagonist, and
ii) a first radiocontrast media,
wherein the pharmaceutical combination is suitable for simultaneous, separate or step-wise administration to humans or mammals.

50. The pharmaceutical combination of claim 49 wherein the first selective adenosine A1 receptor antagonist is administered intravenously in a loading dose followed by subsequent administration as a maintenance dose, wherein the loading dose is administered between about five minutes and about twenty-five minutes prior to the administration of a first radiocontrast media and wherein the maintenance dose is administered over a period of less than about 48 hours subsequent to administration of the loading dose.

51. The pharmaceutical combination of claim 50 wherein the maintenance dose is administered such that the plasma level of the first selective A1 adenosine antagonist is maintained between about 10 ng/ml and about 500 ng/ml.

52. The pharmaceutical combination of claim 49 wherein the first adenosine A1 receptor antagonist is administered orally prior to the administration of a first radiocontrast media.

53. The pharmaceutical combination of claim 49 wherein the first radiocontrast media is not administered until the plasma concentration level of the first adenosine A1 receptor antagonist has reached a concentration of between about 10 ng/ml and about 500 ng/ml.

54. The pharmaceutical combination of claim 49 wherein the first selective adenosine A1 receptor antagonist is selected from pyrrolo[2,3d]pyrimidine derivatives of formula I 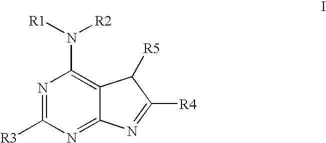
wherein
i) R1 and R2 are each independently selected from a hydrogen atom, an optionally substituted alkyl, optionally substituted aryl, or optionally substituted alkylaryl moiety or together form an optionally substituted heterocyclic ring;
ii) R3 is selected from a hydrogen atom or an optionally substituted alkyl, optionally substituted aryl, or optionally substituted alkylaryl moiety;
iii) R4 and R5 are each independently selected from a halogen atom, a hydrogen atom or an optionally substituted alkyl, optionally substituted aryl, or optionally substituted alkylaryl moiety, or R4 and R5 together form an optionally substituted heterocyclic or optionally substituted carbocyclic ring;
and pharmaceutically acceptable salts of the foregoing, pharmaceutically acceptable prodrugs of the foregoing, and pharmaceutically acceptable solvates of the foregoing.

55. The pharmaceutical combination of claim 54 wherein the first selective adenosine A1 receptor antagonist is selected from pyrrolo[2,3d]pyrimidine derivatives of formula I, wherein
i) R1 and R2 are each independently selected from a hydrogen atom, an optionally substituted alkyl or together form an optionally substituted heterocyclic ring;
ii) R3 is a hydrogen atom or an optionally substituted aryl,
iii) R4 and R5 are each independently selected from a halogen atom or a hydrogen atom;
and pharmaceutically acceptable salts of the foregoing, pharmaceutically acceptable prodrugs of the foregoing, and pharmaceutically acceptable solvates of the foregoing.

56. The pharmaceutical combination of claim 55 wherein the selective adenosine A1 receptor antagonist is selected from pyrrolo[2,3d]pyrimidine derivatives of formula I, wherein
i) R1 is a hydrogen and R2 is an optionally substituted cyclohexyl ring, or R1 and
R2 together form an optionally substituted pyrrolidine ring;
ii) R3 is a phenyl ring,
iii) R4 and R5 are each a hydrogen atom;
and pharmaceutically acceptable salts of the foregoing, pharmaceutically acceptable prodrugs of the foregoing, and pharmaceutically acceptable solvates of the foregoing.

57. The pharmaceutical combination of claim 56 wherein the first selective adenosine A1 receptor antagonist is selected from the group consisting of: 4-[(2-phenyl-7H-pyrrolo[2,3-d]pyrimidin-4-yl)amino]-trans-cyclohexanol methanesulfonate, (4S)-4-hydroxy-1-(2-phenyl-7H-pyrrolo[2,3-o]pyrimidin-4-yl)-L-prolinamide methanesulfonate, pharmaceutically acceptable prodrugs of the foregoing, and pharmaceutically acceptable solvates of the foregoing.

58. The pharmaceutical combination of claim 57 wherein the first selective adenosine A1 receptor antagonist is 4-[(2-phenyl-7H-pyrrolo[2,3-d]pyrimidin-4-yl)amino]-trans-cyclohexanol methanesulfonate, pharmaceutically acceptable prodrugs of the foregoing, and pharmaceutically acceptable solvates of the foregoing.

59. The pharmaceutical combination of claim 57 wherein the first selective adenosine A1 receptor antagonist is (4S)-4-hydroxy-1-(2-phenyl-7H-pyrrolo[2,3-o]pyrimidin-4-yl)-L-prolinamide methanesulfonate, pharmaceutically acceptable prodrugs of the foregoing, and pharmaceutically acceptable solvates of the foregoing.

60. The pharmaceutical combination of claim 50 wherein the first radiocontrast media is an iodinated or gadolinium-based radiocontrast media selected from the group consisting of bunaiod, biligram, bilimiro, bilopaque, cholimil, ethiodol, diatrast, dionosil, falignost, gadobutrol, gadodiamide, gadopentetate dimeglumine, gastrografin, hexabrix, hippodin, mangafodipir, amidotrizoate, ethiodized oil, imagopaque, iodamide, iodipamide, iodixanol, iodophene, iophendylate, iomeron, iomeprol, iopamidol, iopanoic acid, iopiperidol, iophendylate, iopromide, iopydol, iosimenol, iothalamic acid, iotrolan, ioversol, ioxilan, ioxaglic acid, isopaque, ipodate, meglumine iothalamate, meglumine acetrizoate, meglumine diatrizoate, metrizamide, myelotrast, omnipaque, osbil, optiray, optojod, opacoron, perflutren, phenobutiodil, phentetiothalein sodium, priodax, propyliodone, skiodan, sodium iodomethamate, sodium diatrizoate, telepaque, teridax, tetrabrom, thorotrast, triognost, 1,3,5-Tri-n-hexyl-2,4,6-triiodobenzene, tyropanoate, visipaque or xenetix, pharmaceutically acceptable salts of any of the foregoing, prodrugs of any of the foregoing, and solvates of any of the foregoing.

61. A kit comprising
i) a therapeutically effective amount of a first selective adenosine A1 antagonist, and
ii) a first radiocontrast media,
wherein the pharmaceutical combination is suitable for simultaneous, separate or step-wise administration to humans or mammals.

62. The kit of claim 61 further comprising
i) a loading dose of the first selective adenosine A1 receptor antagonist to be administered intravenously; and
ii) a maintenance dose of the first selective adenosine A1 receptor antagonist to be administered intravenously,
wherein the loading dose is administered intravenously followed by subsequent intravenous administration of the maintenance dose, wherein the loading dose is administered between about five minutes and about twenty-five minutes prior to the administration of the first radiocontrast media and wherein the maintenance dose is administered over a period of less than about 48 hours subsequent to administration of the loading dose.

63. The kit of claim 61 wherein the first selective adenosine A1 receptor antagonist is administered intravenously in a loading dose followed by subsequent administration as a maintenance dose, wherein the loading dose is administered between about five minutes and about twenty-five minutes prior to the administration of the first radiocontrast media and wherein the maintenance dose is administered over a period of less than about 48 hours subsequent to administration of the loading dose.

64. The kit of claim 63 wherein the maintenance dose is administered such that the plasma level of the first selective A1 adenosine antagonist is maintained between about 10 ng/ml and about 500 ng/ml.

65. The kit of claim 61 wherein the first adenosine A1 receptor antagonist is administered orally prior to the administration of a first radiocontrast media.

66. The kit of claim 61 wherein the first radiocontrast media is not administered until the plasma concentration level of the first adenosine A1 receptor antagonist has reached a concentration of between about 10 ng/ml and about 500 ng/ml.

67. The kit of claim 61 wherein the first selective adenosine A1 receptor antagonist is selected from pyrrolo[2,3d]pyrimidine derivatives of formula I 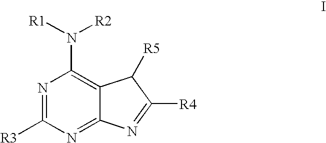
wherein
i) R1 and R2 are each independently selected from a hydrogen atom, an optionally substituted alkyl, optionally substituted aryl, or optionally substituted alkylaryl moiety or together form an optionally substituted heterocyclic ring;
ii) R3 is selected from a hydrogen atom or an optionally substituted alkyl, optionally substituted aryl, or optionally substituted alkylaryl moiety;
iii) R4 and R5 are each independently selected from a halogen atom, a hydrogen atom or an optionally substituted alkyl, optionally substituted aryl, or optionally substituted alkylaryl moiety, or R4 and R5 together form an optionally substituted heterocyclic or optionally substituted carbocyclic ring;
and pharmaceutically acceptable salts of the foregoing, pharmaceutically acceptable prodrugs of the foregoing, and pharmaceutically acceptable solvates of the foregoing.

68. The kit of claim 67 wherein the first selective adenosine A1 receptor antagonist is selected from pyrrolo[2,3d]pyrimidine derivatives of formula i, wherein
i) R1 and R2 are each independently selected from a hydrogen atom, an optionally substituted alkyl or together form an optionally substituted heterocyclic ring;
ii) R3 is a hydrogen atom or an optionally substituted aryl,
iii) R4 and R5 are each independently selected from a halogen atom or a hydrogen atom;
and pharmaceutically acceptable salts of the foregoing, pharmaceutically acceptable prodrugs of the foregoing, and pharmaceutically acceptable solvates of the foregoing.

69. The kit of claim 68 wherein the selective adenosine A1 receptor antagonist is selected from pyrrolo[2,3d]pyrimidine derivatives of formula I, wherein
i) R1 is a hydrogen and R2 is an optionally substituted cyclohexyl ring, or R1 and
R2 together form an optionally substituted pyrrolidine ring;
ii) R3 is a phenyl ring,
iii) R4 and R5 are each a hydrogen atom;
and pharmaceutically acceptable salts of the foregoing, pharmaceutically acceptable prodrugs of the foregoing, and pharmaceutically acceptable solvates of the foregoing.

70. The kit of claim 69 wherein the first selective adenosine A1 receptor antagonist is selected from the group consisting of: 4-[(2-phenyl-7H-pyrrolo[2,3-d]pyrimidin-4-yl)amino]-trans-cyclohexanol methanesulfonate, (4S)-4-hydroxy-1-(2-phenyl-7H-pyrrolo[2,3-d]pyrimidin-4-yl)-L-prolinamide methanesulfonate, pharmaceutically acceptable prodrugs of the foregoing, and pharmaceutically acceptable solvates of the foregoing.

71. The kit of claim 70 wherein the first selective adenosine A1 receptor antagonist is 4-[(2-phenyl-7H-pyrrolo[2,3-d]pyrimidin-4-yl)amino]-trans-cyclohexanol methanesulfonate, pharmaceutically acceptable prodrugs of the foregoing, and pharmaceutically acceptable solvates of the foregoing.

72. The kit of claim 70 wherein the first selective adenosine A1 receptor antagonist is (4S)-4-hydroxy-1-(2-phenyl-7H-pyrrolo[2,3-o]pyrimidin-4-yl)-L-prolinamide methanesulfonate, pharmaceutically acceptable prodrugs of the foregoing, and pharmaceutically acceptable solvates of the foregoing.

73. The kit of claim 61 wherein the first radiocontrast media is an iodinated or gadolinium-based radiocontrast media selected from the group consisting of bunaiod, biligram, bilimiro, bilopaque, cholimil, ethiodol, diatrast, dionosil, falignost, gadobutrol, gadodiamide, gadopentetate dimeglumine, gastrografin, hexabrix, hippodin, mangafodipir, amidotrizoate, ethiodized oil, imagopaque, iodamide, iodipamide, iodixanol, iodophene, iophendylate, iomeron, iomeprol, iopamidol, iopanoic acid, iopiperidol, iophendylate, iopromide, iopydol, iosimenol, iothalamic acid, iotrolan, ioversol, ioxilan, ioxaglic acid, isopaque, ipodate, meglumine iothalamate, meglumine acetrizoate, meglumine diatrizoate, metrizamide, myelotrast, omnipaque, osbil, optiray, optojod, opacoron, perflutren, phenobutiodil, phentetiothalein sodium, priodax, propyliodone, skiodan, sodium iodomethamate, sodium diatrizoate, telepaque, teridax, tetrabrom, thorotrast, triognost, 1,3,5-Tri-n-hexyl-2,4,6-triiodobenzene, tyropanoate, visipaque or xenetix, pharmaceutically acceptable salts of any of the foregoing, prodrugs of any of the foregoing, and solvates of any of the foregoing.

74. A method of using a selective adenosine A1 antagonist comprising:
creating a kit containing a therapeutically effective amount of a first selective adenosine A1 antagonist and a first radiocontrast media.

75. A method of using a selective adenosine A1 antagonist comprising:
administering a therapeutically effective amount of a first selective adenosine A1 antagonist to prevent radiocontrast media induced nephropathy.

76. A method of using a selective adenosine A1 antagonist comprising:
administering a therapeutically effective amount of a first selective adenosine A1 antagonist to prevent a radiocontrast media induced increase in serum creatinine levels.

77. A method of using a selective adenosine A1 antagonist comprising:
administering a therapeutically effective amount of a first selective adenosine A1 antagonist to prevent a radiocontrast media induced decrease in renal blood flow.

78. A method of using a selective adenosine A1 antagonist comprising:
administering a therapeutically effective amount of a first selective adenosine A1 antagonist to prevent or reduce the need of dialysis in a human or mammalian patient receiving a first radiocontrast media.
